(19) United States
(12) Patent Application Publication
(10) Pub. No.: US 2025/0161424 A1
(43) Pub. Date: May 22, 2025

(54) IN OVO VACCINE COMPOSITIONS AGAINST COCCIDIOSIS

(71) Applicant: HIPRA SCIENTIFIC, S.L.U., AMER (ES)

(72) Inventors: MARC PAGES BOSCH, AMER (ES); EDUARD SEBASTIA PUJOL, AMER (ES); MARTA SITJÀ I ARNAU, AMER (ES)

(21) Appl. No.: 18/843,109

(22) PCT Filed: Mar. 7, 2023
(86) PCT No.: PCT/EP2023/055732

§ 371 Date: Aug. 30, 2024

(30) Foreign Application Priority Data
Mar. 8, 2022 (EP) 22382216.4

(51) Int. Cl. A61K 39/002; A61K 39/00; A61K 39/12; A61P 37/04

(52) U.S. Cl. CPC A61K 39/002; A61K 39/12; A61P 37/04; A61K 2039/54; A61K 2039/552

(57) ABSTRACT

A composition, an in ovo vaccine, and a composition for use as a vaccine against coccidiosis. The compositions and vaccine include Eimeria oocysts from a precocious attenuated strain of E. acervulina, and from at least one precocious attenuated strain selected from E. maxima, E. praecox and E. tenella. The composition and vaccine can be administered in ovo to embryonated eggs of a domesticated bird. A combination vaccine for use in the prevention and/or treatment of an Eimeria infection and Infectious Bursal Disease (Gumboro disease) and a kit for use as a vaccine including the composition.

This application claims the benefit of European Patent Application EP22382216 filed on Mar. 8, 2022.

### TECHNICAL FIELD

The present invention belongs to the field of avian coccidiosis caused by Eimeria species. More particularly, the present invention relates to a composition, an in ovo vaccine, and a composition for use as a vaccine against coccidiosis in the prevention and/or treatment of Eimeria infections by in ovo injection. The present invention is further directed to said vaccine for use for generating an immunogenic and/or protective immune response against at least Eimeria infections and kits comprising one or more doses of said vaccine.

### BACKGROUND ART

Avian coccidiosis is one of the most important poultry diseases largely caused by a variety of different Eimeria species parasites, which has been recognized as an economically significant disease of chickens, and associated with considerable economic losses to poultry farmers worldwide. Such great costs, along with the increasing demand for poultry, emphasize the importance of finding new ways to reduce the incidence of coccidiosis in chickens, as disclosed in Blake et al., Re-calculating the cost of coccidiosis in chickens, Vet Res., 2020, 51, 115. As disclosed in Soutter et al., Poultry coccidiosis: design and interpretation of vaccine studies, Frontiers Vet. Sci., 2020, 7, Article 101, Eimeria infection has also been demonstrated to exacerbate the outcome of exposure to other pathogens such as Clostridium perfringens, combining to cause necrotic enteritis.

Coccidiosis has a complex monoxenous life cycle, which consists of both asexual and sexual stages. The cycle is initiated when birds ingest sporulated oocysts, generally associated with faecal material. These oocysts contain the invasive asexual sporozoites, which are released into the bird's digestive tract. The sporozoites then invade epithelial cells, and develop into multinucleate structures called schizonts. Each schizont matures and releases numerous invasive asexual structures, known as merozoites, into the bird's digestive tract, where they in turn invade other epithelial cells. These multiple invasive asexual stages, involving both sporozoites and merozoites, produce the pathological digestive tract lesions characteristic of coccidiosis. The sexual stage of the coccidiosis life cycle is initiated when merozoites differentiate into gametocytes. Gametocytes then fuse and the fertilization products, called oocysts, are released in intestinal lumen and are excreted through the faeces. The release of immature non-sporulated oocysts and its sporulation to form mature sporulated oocysts outside the host completes the parasite's life cycle.

Various species of Eimeria infect a wide range of hosts, including mammals, but seven species of Eimeria (E. acervulina, E. brunetti, E. maxima, E. mitis, E. necatrix, E. praecox and E. tenella) are recognized as affecting chickens, as disclosed in Acharya et al., Alternatives to fight against coccidiosis: a review, Nepalese, Vet. J., 2017, 34, 152-167. Other species of Eimeria that are disclosed to also affect chicken are E. mivati, E. lata, E. nagambie and E. zaria. In chickens, the life cycle of Eimeria tenella, a representative species, is completed in about seven to nine days.

Several approaches have been proposed in the art to fight against coccidiosis.

One approach consists of the application of anticoccidials, which, however, is associated with some degree of resistance to said drugs.

Another approach is the use of vaccines. Immunoprophylaxis designed to fight avian coccidiosis appeared in the United States in the 1950s, when non-attenuated live parasite vaccine began to be used to fight E. tenella. The main problem with this type of vaccines containing non-attenuated parasites lies in the fact that they must be used jointly with anticoccidial products in order to prevent the animals from suffering coccidiosis due to the vaccination itself.

Efficacy of vaccines may be assessed by parameters such as reduction of intestinal lesions, decreased parasite load or replication, lowering environmental oocyst occurrence and thus transmission, reduced clinical signs of disease, and improved performance, whose assessment includes parameters such as feed conversion efficiency, body growth rate, flock homogeneity, and/or egg production.

The administration of a live vaccine seeks the provision of protective immunity by inducing a deliberate infection through the administration of low numbers of Eimeria parasites to the chickens.

Available vaccines against coccidiosis are divided in two main groups: live non-attenuated and live attenuated.

Vaccination is generally performed in the hatchery or farm on the day of the bird's birth by administering the live Eimeria vaccine directly onto the birds by spraying them for example or through its application over their feed and/or drinking water. The infective oocysts complete their life cycle inside the intestinal tract of the bird, as described above, ending with the release of a new generation of non-sporulated oocysts in five to eleven days, depending on the Eimeria species. The non-sporulated oocysts excreted with the bird's faeces then become infective, i.e., sporulate, in the outside environment, and they reinfect the birds through host ingestion. Following two or three such cycles, the birds become immunized against coccidiosis. The acquired immunity wanes over a three to four-month time period in the absence of subsequent exposure to infective oocysts.

Wild-type Eimeria are generally isolated from occurrences of clinical disease in poultry flocks and may be propagated for use as pathogenic challenge strains. Typical non-attenuated vaccines are composed of infective oocysts from mildly to moderately pathogenic strains of the different Eimeria species that have been maintained by laboratory passage. These non-attenuated Eimeria are capable of causing coccidiosis when ingested in high numbers. After the initial dose, the vaccination process relies solely on re-infection through the host's ingestion of sporulated oocysts from the utter. A main concern on the use of wild-type vaccines is the need of administering anticoccidial drugs to stop the replication of the wild-type Eimeria strains.

First commercial vaccine against coccidiosis was based on oral administration of Eimeria species either in drinking water or adding the vaccine in the food of the animals. The first vaccine contained live, non-attenuated Eimeria tenella oocysts, which was further developed by incorporating various Eimeria species, as disclosed in R. B. Williams, Fifty years of anticoccidial vaccines for poultry (1952-2002), Avian Dis., 2002, 46(4), 775-802. However, as disclosed in Soutter et al., op. cit., the use of wild-type, and thus fully virulent, vaccines imply a significant risk, because large number of oocysts accumulate rapidly within chicken house litter resulting in high levels of exposure and clinical disease, even mortality, needing the use of anticoccidial drugs following vaccination.

International patent application WO-A-96/40233 and US patent application US-A-2002/0090378 disclose in ovo live vaccines comprising an effective immunizing dose of live Eimeria sporocysts or oocysts, or a mixture thereof. A vaccine covered by said patent documents is, for example, INOVOCOX EM1 vaccine, a non-attenuated coccidiosis vaccine that contains live oocysts of Eimeria acervulina, Eimeria maxima, and Eimeria tenella, as disclosed in Sokale et al., Effects of administration of an in ovo coccidiosis vaccine at different embryonic ages on vaccine cycling and performance of broiler chickens, Poultry Sci., 2020, 100:100914. However, in Sokale et al., Effects of coccidiosis vaccination administered by in ovo injection on the hatchability and hatching chick quality of broilers, Poult. Sci., 2017, 96, 541-7, it is disclosed that the vaccination in ovo using Inovocox EM1 vaccine results in a decrease in hatchability of injected eggs in comparison to the non-injected treatment group.

International patent application WO-A-96/40234 discloses in ovo live vaccines comprising an effective immunizing dose of live Eimeria sporozoites or merozoites, or a mixture thereof, respectively.

Attenuated vaccines are made up of infective oocysts that have reduced pathogenicity. Due to the strong correlation between attenuated pathogenicity and possession of a shorter prepatent period, many attenuated strains are also precocious. Consistently, attenuated lines that possess shortened prepatent periods are commonly termed “precocious lines”.

Live attenuated vaccines are prepared from Eimeria parasites, which most of them have been selected for an abbreviated lifecycle termed “precocious development”, an attenuation method disclosed in T. K. Jeffers, Attenuation of Eimeria tenella through selection for precociousness, J. Parasitol., 1975, 61(6), 1083-1090. Precocious parasite lines exhibit reduced pathogenicity but retain their immunogenicity. However, the production of attenuated vaccines has not fully met the needs of industrial aviculture. Production of this type of vaccines is complex and the cost is high in comparison to anticoccidial agents and non-attenuated live parasite vaccines. Furthermore, these products have an extremely short lifetime which makes the logistic complex. The difficulty to attenuate all the pathogenic species of Eimeria and the complexity of production, with a large amount of labour and animal handling involved, has led to few products of this type in the market and their use is still limited. Said live attenuated vaccines usually comprise a mixture of sporulated oocysts of different Eimeria species. Attenuated vaccines, however produce fewer oocysts than non-attenuated strains, resulting in slower accumulation of infective oocysts in the environment, which results in a longer time required for the immunization to be fully established.

International patent application WO-A-99/50387 discloses a vaccine which includes one or more attenuated strains of E. maxima ARI-73/97, E. acervulina ARI-77/97, E. tenella ARI-II/98, E. necatrix MCK01 and/or E. necatrix ARIMEDNEC3+8, or antigens of said one or more strains, in association with a veterinary acceptable carrier or excipient administered to hatched chicks.

Vaccines may be administered in drinking water, in an edible gel, sprayed onto feed, spraying over the animals, intra yolk sac injection, or in ovo prior to hatching. In particular, in ovo vaccination include the capability of more rapidly vaccinating a larger number of eggs, a reduction in labour costs and the risks of human error, as disclosed in E. D. Pebbles, In ovo applications in poultry: A review, Poult. Sci., 2018, 97, 2333-38.

However, in ovo vaccine have still different challenges to overcome to be efficient providing immunity. One challenge to be solved by the in ovo administration is associated with a reduction in the hatchability rate for this particular route of administration due to an increase of egg breakage, damage to the embryo or support structures within the egg, and microbial challenge that usually results in late dead and cull bird.

In European patent application EP-A-0291173 it is disclosed a method for controlling coccidiosis, which comprises administering a nonreplicating immunogen effective for inducing immunity against the disease to the bird, wherein the immunogen is administered to the bird while the bird is an embryo enclosed within an egg. In a preferred embodiment, the immunogen is a sporulated Eimeria oocyst extract.

In European patent application EP-A-0650733 it is disclosed a live in ovo vaccine for avian species comprising an attenuated or avirulent microorganism, preferably a specific Salmonella typhimurium strain, which expresses one or more antigen epitopes from a species of Eimeria.

In International patent application WO-A-2009/148895 it is disclosed a vaccine comprising a first strain of an Eimeria species and a second strain of said Eimeria species, wherein the first strain and the second strain have an asynchronous prepatent period; and wherein the first strain is a non-attenuated strain and the second strain is a precocious strain.

Despite the various approaches available in the state of the art, unfortunately existing coccidia vaccines show disadvantages and drawbacks to solve.

Consequently, there is still a need in the art to develop and provide new vaccine compositions against coccidiosis for administering to embryonated eggs with improved cost-efficiency production and with optimal safety and efficacy when compared to compositions based on non-attenuated Eimeria sp. as a new advance in the control of avian coccidiosis.

Furthermore, other diseases affecting avian species, such as Infectious Bursal Disease (IBD) also known as Gumboro disease, may affect hatcheries and farms together with Eimeria infections. Infectious Bursal Disease is an immunosuppressive viral infection caused by infectious bursal disease virus (IBDV) that affects young chicks. IBD is widespread among domestic poultry. IBDV produces histopathological changes in both the bursa and thymus that may affect both humoral and cell-mediated responses. Studies have shown that prior exposure to IBDV may increase the susceptibility to other important poultry diseases, including Eimeria infections, as disclosed in J. J. Giambrone, Effects of infectious bursal disease on the severity of Eimeria tenella infections in broiler chicks, Poult. Sci., 1977, 56(1), 243-6.

Therefore, there is also a need in the art to provide new tools to prevent and control both, Eimeria sp. and IBDV infections in chicks form the first day of life.

### OBJECT OF THE INVENTION

The object of the present invention is a composition for use as a vaccine in the prevention and/or treatment of an Eimeria infection to embryonated eggs of a domesticated birds by in ovo injection.

Another aspect of the invention is said composition for use as a combination vaccine in the prevention and/or treatment of an Eimeria infection and Infectious Bursal Disease.

Another aspect of the invention is a kit for use as a vaccine.

Another aspect of the invention is a composition comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose, and wherein the composition is suitable for preparing an in ovo vaccine.

Another aspect of the invention is a composition comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose, and wherein the composition is suitable for preparing an in ovo vaccine; particularly, with the proviso that the composition does not comprise E. mitis sporulated oocysts.

Another aspect of the invention is an in ovo vaccine comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose.

Another aspect of the invention is an in ovo vaccine comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose; particularly, with the proviso that the in ovo vaccine does not comprise E. mitis sporulated oocysts.

Another aspect of the invention is a kit comprising the composition or the in ovo vaccine of the invention.

### FIGURES

FIG. 1 shows efficacy results of a vaccine composition based on attenuated Eimeria oocysts administered by in ovo injection, as disclosed in Example 2. The mean intestinal lesion score of birds after experimental infection, in vaccinated Groups 1 to 4 (G1 to G4), per Eimeria specie of the experimental infection (E. acervulina (A), E. praecox (P), E. maxima (M) and E. tenella (T)) is depicted. Groups 1 to 3 (G1 to G3) correspond to birds vaccinated with an attenuated Eimeria vaccine comprising different oocysts doses of E. acervulina by in ovo route; Group 4 (G4) corresponds to birds vaccinated with a mock vaccine by in ovo route. The mean intestinal lesion score is represented on the y-axis. Groups 1 to 4 (G1 to G4) and Eimeria species are represented on the x-axis.

FIG. 2 shows safety results of a vaccine composition based on attenuated Eimeria oocysts administered by in ovo injection as disclosed in Example 3. The mean of the body weight (in grams) of birds on days 0, 4.5, 6, 7 and 14 after hatching is represented on the y-axis for Groups 1 to 3 (G1 to G3). Group 1 (G1) corresponds to birds vaccinated with a single dose of an attenuated vaccine composition by in ovo injection. Group 2 (G2) corresponds to birds vaccinated with a ten-fold dose of an attenuated vaccine composition by in ovo injection. Group 3 (G3) corresponds to birds vaccinated with a mock-vaccine composition comprising PBS exclusively. Days after hatch are represented on the x-axis.

FIG. 3 shows the results on the hatchability rate obtained with a vaccine composition based on attenuated Eimeria oocysts administered by in ovo route, as disclosed in Example 4. Hatching rate percentage is represented on the ordinates (y-axis) for each Group (A: control group, and B: vaccinated group). FIG. 3A shows the hatching rate percentage obtained in a hatchery A; FIG. 3B shows the hatching rate percentage obtained in a hatchery B; FIG. 3C shows the overall mean of the hatching rate percentage of the two different hatcheries included in the study.

FIG. 4 shows the results on the efficacy of one dose of the combination of an attenuated Eimeria vaccine against avian coccidiosis and a live attenuated vaccine against Infectious Bursal Disease (IBD) by in ovo administration to embryonated chicken eggs against a challenge with very virulent IBD virus (vvIBDv), as disclosed in Example 5. The mean value of the acute functional histopathological lesion score 6 days after the experimental infection (day 30 of the study) is depicted on the ordinates, for each group of treatment (Group 1: vaccinated group, and Group 2: control group) on the abscissas.

FIG. 5 shows further results of Example 5, wherein the mean value of the mononuclear infiltration score 6 days after the experimental infection (day 30 of the study) is depicted on the ordinates, for each group of treatment (Group 1: vaccinated group, and Group 2: control group) on the abscissas.

FIG. 6 shows further results of Example 5, wherein the mean value of the plical oedema score 6 days after the experimental infection (day 30 of the study) is depicted on the ordinates, for each group of treatment (Group 1: vaccinated group, and Group 2: control group) on the abscissas.

FIG. 7 shows further results of Example 5, wherein the mean value of the oedema of muscular wall score 6 days after the experimental infection (day 30 of the study) is depicted on the ordinates, for each group of treatment (Group 1: vaccinated group, and Group 2: control group) on the abscissas.

FIG. 8 shows further results of Example 5, wherein the mean value of the serosal oedema score 6 days after the experimental infection (day 30 of the study) is depicted on the ordinates, for each group of treatment (Group 1: vaccinated group, and Group 2: control group) on the abscissas.

FIG. 9 shows the results on the efficacy of one dose of the combination of an attenuated Eimeria vaccine against avian coccidiosis and a live attenuated vaccine against Infectious Bursal Disease (IBD) by in ovo administration to embryonated chicken eggs against a challenge with four different Eimeria spp. The mean value of the intestinal lesion score of birds after experimental infection per Eimeria species, is depicted on the ordinates, for each group of treatment (Group 1: vaccinated group, and Group 2: control group) and for each Eimeria specie (E. acervulina: A; E. maxima: B; E. praecox: C; and E. tenella: D), on the abscissas.

FIG. 10, related to Example 7, shows the average Eimeria spp. oocysts count in fresh faeces (oocysts/gram of fresh faeces) depicted on the ordinates, from day 1 to day 9 post-hatch on the abscissas, for each group of treatment (Group 1 (solid line): live attenuated Eimeria spp. vaccine composition of the invention administered in ovo, and Group 2 (dashed line): live attenuated Eimeria spp. vaccine administered by coarse spray post-hatch).

### DETAILED DESCRIPTION OF THE INVENTION

The object of the present invention is a composition, an in ovo vaccine, and a composition for use as a vaccine in the prevention and/or treatment of an Eimeria infection, wherein the composition comprises Eimeria oocysts from a precocious attenuated strain of E. acervulina, and from at least one precocious attenuated strain selected from E. maxima, E. praecox and E. tenella, wherein an effective immunizing dose of the vaccine is administered in ovo to embryonated eggs of a domesticated bird. Another wording of the object of the invention is a composition, an in ovo vaccine, and a composition for use as a vaccine in the prevention and/or treatment of an Eimeria infection, wherein the composition comprises Eimeria oocysts from a precocious attenuated strain of E. acervulina, and from at least one precocious attenuated strain selected from E. maxima, E. praecox and E. tenella, wherein the vaccine is administered in ovo to embryonated eggs of a domesticated bird.

Another object of the invention is an in ovo vaccine comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose, with the proviso that the in ovo vaccine does not comprise E. mitis sporulated oocysts.

Another object of the invention is a method in the prevention and/or treatment of an Eimeria infection by administering in ovo an effective immunizing dose of a composition that comprises Eimeria oocysts from a precocious attenuated strain of E. acervulina, and from at least one precocious attenuated strain selected from E. maxima, E. praecox and E. tenella to embryonated eggs of a domesticated bird. In an embodiment, said prevention and/or treatment results in an improvement in comparison to a non-treated control group of the same domesticated bird.

The authors of the present invention have surprisingly found a composition comprising oocysts from definite attenuated species of Eimeria, which can effectively induce an immune response, particularly a protective immune response against an Eimeria infection by administration in ovo. Additionally, the inventors have surprisingly observed an increase in the hatching rate of the in ovo vaccinated eggs contrary what the prior art discloses.

The authors also unexpectedly found that a combination vaccine comprising a composition comprising oocysts from attenuated strains of Eimeria in combination with a composition comprising IBDV (Infectious Bursal Disease Virus) antigens administered by in ovo route was safe and elicited an effective protection against both, Eimeria sp. and IBDV infections at the same time.

In the present description, as well as in the claims, the singular forms “a”, “an” and “the” include the plural reference unless the context clearly indicates otherwise. The ranges defined by the terms “between . . . and . . . ” or by the terms “from . . . to . . . ” include also the two ends thereof. The term “about” refers to a deviation of plus/minus 10%, preferably plus/minus 5%.

In the scope of the present invention, the term “vaccine”, “vaccine composition” and “a composition for use as a vaccine” refers to a preparation that is administered to stimulate the subject's immune response against a specific infectious disease, in particular, a preparation for the prevention and/or treatment of an Eimeria infection. The term “vaccine”, “vaccine composition” and “a composition for use as a vaccine” also used herein, refers to a preparation that elicits an immunological response in the host of a cellular or antibody-mediated type upon administration to the subject, in particular a response that is protective. The preparation may contain different Eimeria species that infect domesticated birds. The vaccine and vaccine compositions of the invention are further addressed to prevent and/or treat avian coccidiosis.

The term “domesticated bird(s)”, as used herein, unless otherwise indicated, includes chickens, turkeys, ducks, game birds (including, but not limited to, quail, pheasants, and geese) and ratites (including, but not limited to, ostrich). Preferably, the domesticated birds are poultry, and more preferably are chicken. In the context of the present invention the term “domesticated bird(s)” is also referred as the host or the subject.

The term “in ovo to embryonated eggs”, as used herein, unless otherwise indicated, means into a domesticated bird egg containing a live, developing embryo.

The term “administering in ovo” or “in ovo administration” or “in ovo route” or “in ovo injection”, as used herein, unless otherwise indicated, means administering the vaccine described herein to a domesticated bird egg containing a live, developing embryo by any means of penetrating the shell of the egg and introducing the vaccine. Such means of administration include, but are not limited to, injection of the vaccine. Accordingly, the term “in ovo vaccine” refers to a vaccine suitable for in ovo administration. In ovo administration encompasses the administration of the composition for use as a vaccine of the invention during the final quarter of incubation of the domesticated bird. In chicken embryonated eggs in ovo administration is done on days 15 to 20 of incubation, preferably on day 17 to 19.5 of incubation, more preferably on day 18 to 19.2 of incubation, and yet more preferably at approximately 18.5 days of incubation. In ovo administration can be performed using any suitable in ovo administration method, preferably the vaccine is administered via injection. According to one method of injection, a hole is made in the egg's shell at the large end of the egg using an 18-gauge needle to expose the egg's air cell. A 2.54-3.81 cm (1.0-1.5-inch) 22-gauge needle attached to a syringe of appropriate size can be inserted through the hole and through the membrane of the air cell. The site of injection can be within any region of the egg, preferably injection is done axially through the centre of the large end of the egg into the amnion. Alternatively, automated egg injection systems can also be used such as Embrex® Inovoject Systems (Zoetis Inc.), Egginject® In ovo Systems (Ceva Animal Health), Ovo-Jector® (Boehringer Ingelheim), Select Inject™ (Vinovo Poultry Health Solutions), Ultimate In-ovo Egg Injection (Innovatec Hatchery Automation B.V.), Sanovo vax (Sanovo Technology Group). Other appropriate methods of injection are known to those skilled in the art.

The term “oocysts”, as used herein, unless otherwise indicated, means live Coccidia oocysts, particularly means live Eimeria sporulated oocysts or a mixture of sporulated and non-sporulated oocysts.

The term “Eimeria”, as used herein, unless otherwise indicated, means one or more species of the genus Eimeria that infect domesticated birds. Eimeria species include those that are found in chickens, and include, among others E. tenella, E. acervulina, E. maxima, E. necatrix, E. mitis, E. praecox, E. mivati and E. brunetti, and also those that are found in turkeys, including E. meleagrimitis, E. adenoeides, E. gallopavonis, E. dispersa, E. meleagridis, E. innocua, and E. Subrotunda, and also Eimeria species that infect other domesticated birds as defined above. The term “Eimeria” also includes any newly-discovered strains or species of Eimeria that infect domesticated birds as defined above.

The term “attenuated” strain or “attenuated Eimeria strain” as used herein, unless otherwise indicated, means a strain that has been selected for its reduced pathogenicity in the host. Attenuation of virulent or wild-type strains of Eimeria spp. can be achieved by a number of means such as attenuation by repeated passage in chicken embryos to produce embryo-adapted lines, attenuation by selection for early maturation of oocysts during serial passage (such as serial chick or embryo passage) to produce precocious lines, or other means known by the skilled person such as but not limited to chemical mutagenesis, and by irradiation methods. Preferably, the attenuation of Eimeria strains is done by performing a number of serial embryo passages.

The term “precocious” or “precocious attenuated strain”, as used herein, unless otherwise indicated, means an Eimeria strain that has a reduced prepatent period compared to a non-attenuated strain of the same Eimeria species.

The term “wild-type”, as used herein, unless otherwise indicated, means an Eimeria field isolate which has not been altered or modified by attenuating passage or any other attenuation treatment. Wild-type Eimeria strains may have different degrees of pathogenicity.

The term “strain” or “Eimeria strain”, as used herein, unless otherwise indicated, means a subpopulation of the species of the Coccidia genus that can be differentiated from the general population of that species by one or more features, like: genome structure and organization, pathogenicity, immunogenicity, geographical distribution, prepatent period, and/or a population resulting from expansion of a single oocyst.

The term “parent strain” or “parental strain”, as used herein, means a subpopulation of the species of the Coccidia genus that is generated from a pool of population obtained from wild-type oocysts. In particular, a subpopulation of an Eimeria specie that is generated form wild-type oocysts. The parent strain is a wild-type strain, and as such it may have different degrees of pathogenicity. When preparing attenuated strains, the parent strain is considered the original strain from which an attenuation pressure is initiated to finally obtain the attenuated strain. As previously described, attenuation can be achieved by a number of means including serial passaging the parent strain (such as serial chick or embryo passage).

The term “effective immunizing dose” or “immunologically effective amount”, as used herein, unless otherwise indicated, means an amount that induces an immune response in vaccinated animals, in particular means the number of live oocysts sufficient to elicit an immune response in vaccinated animals, and further provides immunological protection to the hatched birds that is greater than the inherent immunity of non-immunized birds and/or non-treated control group of the same domesticate birds. As used herein, the terms “immunize” or “immunisation” and “vaccinate” or “vaccination” are synonymous and are used interchangeably. The effective immunizing dose may be administered either in a single dose or as part of a series, and it is effective for treatment and/or prevention of Eimeria species infections and/or coccidiosis. The effective immunizing dose varies depending upon the health and physical condition of the subject to be treated; age; the capacity of the subject's immune system to produce antibodies from any of the immunological classes, such as immunoglobulins A, D, E, G or M, the proliferation of B and T lymphocytes, the provision of activation, growth and differentiation signals to immunological cells, expansion of helper T cells, suppressor T cells, and/or cytotoxic T cell and/or y, 5-T cell populations; the degree of protection desired; the formulation of the vaccine; the veterinarian's assessment of the medical situation, and other relevant factors. The skilled person is aware for a given vaccine, what is an effective immunizing dose according to the definition by using routine methods well known in the art without exercising any inventive skill, such as, among others, the methods disclosed in example 2 below.

The term “immunological response” or “immune response”, as used herein, unless otherwise indicated, means an immune reaction to an antigen or composition that develops in the vaccinated subject of an innate, humoral and/or a cellular immune response to an antigen present in the composition of interest. Thus, the response elicits a rise in corresponding antibody titers and/or an activation of cell-mediated immunity. Preferably, the immune reaction that is elicited provides protective immunity that prevents and/or reduces clinical disease signs of coccidiosis. Preferably, the immune response prevents and/or reduces clinical signs of coccidiosis such as weight loss, morbidity and/or mortality in the vaccinated subjects, i.e., domesticated birds as defined above, when challenged naturally or experimentally with a virulent dose of an Eimeria species. The prevention and/or reduction of clinical signs of coccidiosis results in an improvement in comparison to an infected non-immunized or non-treated control group of the same domesticated birds. Preferably, said improvement comprises a vaccine efficacy parameter selected from the group consisting of reduction of the body weight loss, reduction of the Eimeria oocyst excretion, reduction of the Eimeria oocysts load, reduction of the Eimeria oocysts shedding, reduction of the Eimeria transmission, depression, ruffled feathers, arched body position, diarrhoea, alteration of faeces with blood or mucous content, dehydration, intestinal lesions, morbidity and/or mortality.

The term “combination” or “combination vaccine”, as used herein, unless otherwise indicated, means that the vaccine contains various antigens in a single preparation. Thus, the composition, the in ovo vaccine, or the composition for use as a vaccine of the invention can be also administered in combination with other vaccine compositions. Preferably the vaccines to be combined are mixed into a single preparation prior to the in ovo administration. Combination vaccine also encompasses vaccines that are not mixed but administered concurrently at the same time to the domesticated bird. Thus, the combination vaccine may be in a single preparation or in a different preparation. The combination vaccine may be in the same container or in a different container.

The term “treatment”, as used herein, unless otherwise indicated, refers to any type of therapy with the immunogenic composition or composition for use as a vaccine of the invention, which is aimed at terminating, preventing, ameliorating or reducing the susceptibility to a clinical condition or existing disease as described herein, including complete curing of a disease as well as amelioration or alleviation of said disease. In a preferred embodiment, the term treatment relates to prophylactic treatment (i.e., a therapy to prevent or reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a subject, including domesticated birds. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or, at least, clinical signs associated therewith, so that the host no longer suffers from the disorder or its clinical signs, such as causing regression of the disorder or its clinical signs, for example, by restoring or repairing a lost, missing or defective function, or stimulating an inefficient process, or (4) relieving, alleviating, or ameliorating the disorder, or clinical signs associated therewith, where ameliorating is used in a broad sense to refer to at least a reduction in the magnitude of a clinical sign parameter, reduction of the pathogen load, the pathogen shedding, or reduction in pathogen transmission.

The term “prevention” or “reduction” or “preventing” or “reducing”, respectively, as used herein, means, but is not limited to, a process of prophylaxis in which a subject is exposed to the immunogenic composition or composition for use as a vaccine of the invention prior to the induction or onset of the disease process, and wherein said immunogenic composition or vaccine composition, when administered to said subject, elicits or is able to elicit an immune response in said subject against Eimeria infections. Altogether, such treatment results in prevention or reduction of the clinical signs of avian coccidiosis or of clinical signs associated with avian coccidiosis, respectively or reduction of Eimeria oocysts output. More specifically, the term “prevention” or “preventing”, as used herein, means generally a process of prophylaxis in which a domesticated bird is exposed to the immunogenic or composition for use as a vaccine of the present invention prior to the induction or onset of avian coccidiosis.

Herein, “reduction of clinical signs associated with avian coccidiosis or Eimeria infection” means, but is not limited to, reducing the number of infected subjects in a group, reducing or eliminating the number of subjects exhibiting clinical signs of Eimeria infection, or reducing the severity of any clinical signs that are present in the subjects, in comparison either to wild-type infected subjects or to experimentally infected non-immunized, i.e., non-treated, subjects. For example, it should refer to any reduction of Eimeria oocysts output, reduction in Eimeria oocysts transmission, or reduction of any clinical sign of avian coccidiosis. Preferably these clinical signs are reduced in subjects receiving the composition for use as a vaccine of the present invention by at least 10 percent (%) in comparison to subjects not receiving the composition and may become infected. More preferably, clinical signs are reduced in subjects receiving the composition for use as a vaccine of the present invention by at least 20 percent (%), preferably by at least 30 percent (%), more preferably by at least 40 percent (%), and even more preferably by at least 50 percent (%) in comparison to subjects not receiving the composition and may become infected. The clinical signs, as mentioned herein, can be selected from the group consisting of depression, ruffled feathers, arched body position, diarrhoea, alteration of faeces with blood or mucous content, dehydration, intestinal lesions, decreased body weight, and Eimeria oocysts excretion.

Herein, “reduction of clinical signs associated with IBDV, Infectious Bursal Disease Virus or Gumboro disease” means, but is not limited to, reducing the number of infected subjects in a group, reducing or eliminating the number of subjects exhibiting clinical signs of IBDV infection, or reducing the severity of any clinical signs that are present in the subjects, in comparison either to wild-type infected subjects or to experimentally infected non-immunized, i.e., or non-treated, subjects. For example, it may refer to any reduction of the clinical signs selected form the group consisting of mean acute histological functional score, inflammatory infiltrate, plical oedema, and absence of serosal oedema and oedema in the muscular wall. Preferably, these clinical signs are reduced in subjects receiving the composition for use as a vaccine of the invention by at least 10 percent (%) in comparison to subjects not receiving the composition of the invention and may become infected. More preferably, the clinical signs are reduced by at least 20 percent (%), even more preferably by at least 30 percent (%), even more preferably by at least 40 percent (%), and even more preferably by at least 50 percent (%) in comparison to subjects not receiving the composition of the invention and may become infected.

### Vaccine Composition

In a first embodiment, the composition for use as a vaccine provides an immunogenic and/or protective immune response against at least an Eimeria infection.

In a further embodiment, the composition for use as a vaccine provides an immunogenic and/or protective immune response against an infection caused by at least one Eimeria species.

In a further embodiment, the composition for use as a vaccine reduces or prevents hatched birds from one or more clinical signs associated with avian coccidiosis.

In a further embodiment, the composition for use as a vaccine reduces or prevents hatched birds from one or more clinical signs associated with avian coccidiosis, wherein the avian coccidiosis is caused by Eimeria infection.

In a further embodiment, the clinical signs are selected from the group consisting of intestinal lesions, decreased body weight, oocysts output, oocysts load, oocysts shedding, diarrhoea, dehydration, presence of blood in faeces, presence of mucous in faeces, Eimeria transmission, depression, ruffled feathers, arched body position, morbidity, and mortality.

In a further embodiment, the clinical signs are selected from the group consisting of intestinal lesions, decreased body weight, oocysts output, diarrhoea, dehydration, presence of blood in faeces and presence of mucous in faeces.

In a further embodiment, the composition for use as a vaccine increases the hatching rate compared to a non-vaccinated control group. In an embodiment, the control group is being administered with PBS.

In a further embodiment, the composition for use as a vaccine of the invention reduces the oocysts output of hatched birds compared to hatched birds vaccinated in ovo with a composition comprising non-attenuated Eimeria strains.

In a further embodiment, the effective immunizing dose of the composition for use as a vaccine of the invention reduces the oocysts output of hatched birds compared to hatched birds vaccinated in ovo with a composition comprising non-attenuated Eimeria strains.

In a further embodiment, the domesticated bird is selected from chickens, turkeys, ducks, game birds and ratites. Preferably, the domesticated birds are chicken.

### Eimeria Infection

In an embodiment, the Eimeria infection causes coccidiosis disease. In an embodiment, said coccidiosis disease is caused by an Eimeria specie selected from the group consisting of E. acervulina, E. maxima, E. praecox, and E. tenella.

In a further embodiment, the composition for use as a vaccine is for the prevention of an Eimeria infection.

In a further embodiment, the composition for use as a vaccine is for the treatment of an Eimeria infection.

In a further embodiment, the composition for use as a vaccine is for the prevention of avian coccidiosis.

In a further embodiment, the composition for use as a vaccine is for the treatment of avian coccidiosis.

### Eimeria Strains

The composition, the in ovo vaccine, or the composition for use as a vaccine of the invention comprises, consists of, or consists essentially of Eimeria oocysts from a precocious attenuated strain of E. acervulina, and from at least one precocious attenuated strain selected from E. maxima, E. praecox and E. tenella.

In an embodiment, the composition, the in ovo vaccine, or the composition for use as a vaccine consists of, or consists essentially of Eimeria oocysts from precocious attenuated strains of E. acervulina and E. maxima.

In an embodiment, the composition, the in ovo vaccine, or the composition for use as a vaccine consists of, or consists essentially of Eimeria oocysts from precocious attenuated strains of E. acervulina and E. praecox.

In an embodiment, the composition, the in ovo vaccine, or the composition for use as a vaccine consists of, or consists essentially of Eimeria oocysts from precocious attenuated strains of E. acervulina and E. tenella.

In a preferred embodiment, the composition, the in ovo vaccine, or the composition for use as a vaccine comprises, consists of, or consists essentially of Eimeria oocysts from a precocious attenuated strain of E. acervulina, and from at least two precocious attenuated strains selected from E. maxima, E. praecox and E. tenella. In an embodiment, the composition, the in ovo vaccine, or the composition for use as a vaccine consists of, or consists essentially of Eimeria oocysts from a precocious attenuated strain of E. acervulina, E. maxima, and E. praecox. In an embodiment, the composition, the in ovo vaccine, or the composition for use as a vaccine consists of, or consists essentially of Eimeria oocysts from a precocious attenuated strain of E. acervulina, E. maxima, and E. tenella. In an embodiment, the composition, the in ovo vaccine, or the composition for use as a vaccine consists of, or consists essentially of Eimeria oocysts from a precocious attenuated strain of E. acervulina, E. praecox and E. tenella.

In another preferred embodiment, the composition, the in ovo vaccine, or the composition for use as a vaccine of the invention comprises, consists of, or consists essentially of a mixture of sporulated oocysts from precocious attenuated strains of E. acervulina, E. maxima, E. praecox, and E. tenella.

In another embodiment, the composition, the in ovo vaccine, or the composition for use as a vaccine of the invention comprises Eimeria oocysts, wherein the Eimeria oocysts are from a precocious attenuated strain of E. acervulina, and from at least one precocious attenuated strain selected from E. maxima, E. praecox and E. tenella. In another embodiment, the composition, the in ovo vaccine, or the composition for use as a vaccine of the invention comprises Eimeria oocysts, wherein the Eimeria oocysts are from a precocious attenuated strain of E. acervulina, E. maxima, E. praecox and E. tenella.

In a further embodiment, the composition, the in ovo vaccine, or the composition for use as a vaccine in the prevention and/or treatment of an Eimeria infection is with the proviso that it does not comprise E. mitis sporulated oocysts.

In a further embodiment, the composition, the in ovo vaccine, or the composition for use as a vaccine in the prevention and/or treatment of an Eimeria infection comprises Eimeria oocysts selected from E. acervulina precocious attenuated strain with reduced pathogenicity and reduced oocysts output, i.e., excretion, in comparison to a parent E. acervulina strain in a period of 14 days.

In a further embodiment, the E. acervulina precocious attenuated strain used in the composition, the in ovo vaccine, or the composition for use as a vaccine of the invention when administered to the host, the host excretes from about 30% to about 75% less, preferably from about 35% to about 65% less, more preferably from about 40% to about 60% less, and yet more preferably from about 45% to about 55% less of oocysts during a period of 14 days compared to the oocysts excreted by a host that received the parent E. acervulina strain.

In a further embodiment, the E. acervulina precocious attenuated strain used in the composition, the in ovo vaccine, or the composition for use as a vaccine of the invention when administered to the host, the host excretes in a period of 14 days a similar number of oocysts as the oocysts excreted by a host that received the E. acervulina strain 120718P5 in a period of 14 days. Preferably, the E. acervulina precocious attenuated strain used in the composition, the in ovo vaccine, or the composition for use as a vaccine of the invention when administered to the host, the host excretes in a period of 14 days about ±75% of the oocysts excreted by a host that received the E. acervulina strain 120718P5, preferably about ±70%, preferably about ±60%, preferably about ±50%, preferably about ±40%, preferably about ±30% of the oocysts excreted by a host that received the E. acervulina strain 120718P5, more preferably about ±25%, more preferably about ±20%, more preferably about ±15% of the oocysts excreted by a host that received the E. acervulina strain 120718P5, and yet more preferably about ±10% of the oocysts excreted by a host that received the E. acervulina strain 120718P5.

Oocysts output and counting may be done by using any method disclosed in the art, for example, in Shirley et al., COST 89/820: Biotechnology: guidelines on techniques in coccidiosis research, 1995, Luxembourg: Office for Official Publications of the European Communities.

In an embodiment, the E. acervulina precocious strain used in the composition, the in ovo vaccine, or the composition for use as a vaccine of the invention is attenuated by repeated passages of selection for precocious development from a parent strain. In a further embodiment, the repeated passages are laboratory repeated passages. In a particular embodiment, the E. acervulina precocious strain is attenuated after performing at least 3 passages, at least 5 passages, at least 6 passages, at least 7 passages, at least 8 passages, at least 9 passages, at least 10 passages, or at least 11 passages of selection for precocious development from the parent strain. In a further embodiment, the E. acervulina precocious strain is attenuated after performing at least 5 passages of selection for precocious development from the parent strain.

In an embodiment, the E. acervulina precocious attenuated strain used in the composition, the in ovo vaccine, or the composition for use as a vaccine of the invention is E. acervulina strain 120718P5, deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021. This strain has a reduced pathogenicity and when administered to the host, the host excretes about 50% less of live oocysts compared to the oocysts excreted by a host that received the parent E. acervulina strain during a period of 14 days. This strain is obtained by laboratory passages, preferably it is obtained after 5 repeated passages of selection for precocious development from the parent strain using a method such as disclosed, for example, in Jeffers et al., op. cit.

Oocysts from precocious attenuated strains of E. maxima, E. praecox, and E. tenella are well known in the art and the skilled person knows how to obtain them. Moreover, there are some commercially available vaccines on the market containing said precocious attenuated strains, for example, the oocysts may be isolated from among other sources through commercial vaccine compositions such as EVANT® (Laboratorios Hipra, S.A., Avda. La Selva 135, 17170 Amer, Girona, Spain) and PARACOX 8® (Merck Sharp & Dohme Animal Health, S.L., 37008 Carbajosa de la Sagrada, Salamanca, Spain), which comprise a combination of Eimeria oocysts, including oocysts of E. maxima, E. praecox, and E. tenella. Other sources of oocysts from precocious attenuated strains of E. maxima and E. tenella are EVALON® (Laboratorios Hipra, S.A., Avda. La Selva 135, 17170 Amer, Girona, Spain), PARACOX 5® (Merck Sharp & Dohme Animal Health, S.L., 37008 Carbajosa de la Sagrada, Salamanca, Spain), HATCHPAK® COCCI III (Boehringer Ingelheim Animal Health USA Inc., 3239 Duluth, USA) and HuveGuard MMAT® (Huvepharma NV., 2600 Antwerp, Belgium).

### Oocysts

Oocysts of precocious attenuated strains of Eimeria to be used according to the invention can be prepared by any of several methods known to those skilled in the art, for instance, Shirley et al., op. cit.

Attenuation of pathogenicity of each Eimeria specie is achieved by repeated passage in chickens with selection for early appearance of oocysts, as disclosed in Jeffers, op.cit. By selection for precocious development, populations can be selected with an important reduction of pre-patent periods (i.e., the time from ingestion of sporulated oocysts to emergence of oocysts in the faeces) and a significant reduction of pathogenicity.

The attenuation of each Eimeria specie involves the inoculation of the purified parent strains to coccidia-free specified pathogen free (SPF) chicks to obtain an initial passage (PO). The first oocysts eliminated from faeces are re-inoculated then to new coccidia-free SPF chicks to perform a second passage. A number of similar successive and repeated passages are performed until populations of oocysts with greatly reduced pre-patent times and pathogenicity compared to the parent strains are obtained.

For the obtention of Eimeria oocysts for the preparation of the composition, the in ovo vaccine, or the composition for use as a vaccine of the invention, coccidia-free SPF chicks are inoculated with a suspension of sporulated oocysts for each Eimeria species to be produced. Their faeces are collected after a concrete period of time and oocysts are recovered. Then, they are sporulated and the resulting oocysts are reinoculated to a new group of coccidia-free SPF chicks. This process is repeated several times. In particular, the process is repeated a further three times, and the oocysts for each Eimeria specie are recovered. The recovered oocysts are finally purified and sporulated.

As known in the prior art patency pattern for each Eimeria sp. may differ depending on each Eimeria species and strains. The completion of the life cycle indicated by the emergence of unsporulated oocysts in the host faeces is known as patency. Therefore, the period of collection of oocysts from bird's faeces may vary and the skilled person is aware of the optimum time for collecting oocysts from each Eimeria species and strains, as disclosed in Shirley et al., op. cit.

The oocysts recovered for each Eimeria species may be sporulated by any means known by the skilled artisan such as potassium dichromate solution, potassium sorbate solution or compositions comprising peroxygen and organic acids among others. The oocysts are sporulated preferably with a 2-3% potassium dichromate solution, by incubating at 29° C. for 48 h with aeration. After the sporulation, the potassium dichromate solution is removed by centrifugation. Sporulated oocysts are disinfected with sodium hypochlorite and once removed, they are resuspended in PBS (phosphate-buffered saline solution).

Then, sporulated oocysts of the different Eimeria species are counted for each bulk suspension, and the volume to be mixed of each Eimeria species' oocysts suspension is calculated. A final suspension is obtained by mixing the different Eimeria oocysts' suspensions and adjusting the final volume with PBS.

Sporocysts obtained from disinfected sporulated oocysts may be frozen (cryopreserved) in liquid nitrogen for storage according to methods known in the art, such as disclosed in Shirley et al., op. cit.

The oocysts of the present invention can be used for preparing compositions, in ovo vaccines, or compositions for use as a vaccine for the prevention and/or treatment of an Eimeria infection. In an embodiment, the oocysts of the invention are used for preparing a composition or an in ovo vaccine with a dose comprising from 10 to 10⁸ oocysts per embryonated egg, from 10 to 107 oocysts per embryonated egg, from 10² to 107 oocysts per embryonated egg, from 10² to 10⁶ oocysts per embryonated egg, or from 10² to 105 oocysts per embryonated egg. More preferably, the composition, the in ovo vaccine, or the composition for use as a vaccine of the invention comprises a dose from 10² to 104 oocysts per embryonated egg.

### Excipients

In an embodiment the in ovo vaccine, or the composition for use as a vaccine of the invention comprises at least a pharmaceutically or veterinary acceptable carrier or vehicle or excipient or adjuvant.

The pharmaceutically or veterinary acceptable carrier or vehicle or excipients may be any compound or combination of compounds facilitating the administration and/or delivery of the composition or the in ovo vaccine. Among other compounds it can include for example wetting agents, dispersant agents, emulsifying agents, buffer agents (for example phosphate buffer), stabilizing agents such as carbohydrates (for example glucose, sucrose, mannitol, sorbitol, starch or dextrans) or proteins (for example, albumin, casein, bovine serum or skimmed milk).

The pharmaceutically or veterinary acceptable carriers or vehicles are well known to the one skilled in the art, and may include, among other components, water, saline, buffered saline, such as phosphate buffered saline, or any other physiologically acceptable medium.

In an embodiment, the composition, the in ovo vaccine, or the composition for use as a vaccine includes wetting agents, dispersant agents, emulsifying agents, buffer agents, salts, stabilizing agents, proteins, water, saline (e.g., sodium chloride and/or potassium chloride solutions), buffered saline (e.g., phosphate buffer), physiologically acceptable medium, and mixtures thereof.

Suitable excipients for the composition or the in ovo vaccine of the invention may be found, for example, in the book Handbook of pharmaceutical excipients, R. C. Rowe, P. J. Sheskey and M. E. Quinn, editors, Pharmaceutical Press, sixth edition, 2009.

In an embodiment, a pharmaceutically or veterinary acceptable carrier or vehicle or excipient can be selected from the group of sodium chloride or potassium chloride, a phosphate buffer (e.g., a combination of disodium phosphate and potassium dihydrogen phosphate), a surfactant, preferably non-ionic surfactant (e.g., polysorbates, which are ethoxylated sorbitan fatty acid esters), and mixtures thereof.

In an embodiment, the composition, the in ovo vaccine, or the composition for use as a vaccine of the invention is an aqueous suspension. In a further embodiment, the composition, the in ovo vaccine, or the composition for use as a vaccine comprises as vehicle and excipients a combination of disodium phosphate, potassium dihydrogen phosphate, polysorbate, potassium chloride, sodium chloride, and water.

In a further embodiment, the composition, the in ovo vaccine, or the composition for use as a vaccine of the invention comprises polysorbate at a concentration ranging from about 0.001% to about 1% (v/v), from about 0.005% to about 0.5% (v/v), from about 0.006% to about 0.3% (v/v), from about 0.007% to about 0.1% (v/v), from about 0.005% to about 0.05% (v/v). Preferably, the composition, the in ovo vaccine, or the composition for use of the invention comprises polysorbate at a concentration of about 0.005%, 0.006%, 0.007%, 0.008%, 0.009%, 0.01%, 0.02%, 0.03%, 0.04%, 0.05%, 0.1%, (v/v). More preferably, the composition, the in ovo vaccine, or the composition for use as a vaccine of the invention comprises polysorbate at a concentration of about 0.01% and 0.02% (v/v), even more preferably of about 0.01% (v/v).

In a further embodiment, the composition, the in ovo vaccine, or the composition for use as a vaccine of the invention comprises a polysorbate selected from the group consisting of polysorbate 20 (polyoxyethylene 20 sorbitan monolaurate), polysorbate 40 (polyoxyethylene 20 sorbitan monopalmitate), polysorbate 60 (polyoxyethylene 20 sorbitan monostearate), and polysorbate 80 (polyoxyethylene 20 sorbitan monooleate). Preferably, the composition, the in ovo vaccine, or the composition for use as a vaccine of the invention comprises polysorbate 80.

The composition, the in ovo vaccine, or the composition for use as a vaccine of the invention may comprise or consist essentially of one or more adjuvants. Suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one or more non-methylated CpG units, (3) an oil in water emulsion, (4) cationic lipids containing a quaternary ammonium salt, (5) cytokines, (6) aluminium hydroxide or aluminium phosphate or (7) any combinations or mixtures thereof.

Among the type (1) adjuvant polymers, preference is given to polymers of crosslinked acrylic or methacrylic acid, especially crosslinked by polyalkenyl ethers of sugars or polyalcohols. These compounds are known under the name carbomer. Products sold under the name Carbopol (BF Goodrich, Ohio, USA).

Advantageously, the mixture with the adjuvant is formed extemporaneously and preferably contemporaneously with administration of the preparation or shortly before administration of the preparation; for instance, shortly before or prior to administration, the vaccine-adjuvant mixture is formed, advantageously so as to give enough time prior to administration for the mixture to form a complex, e.g., between about 10 and about 60 minutes prior to administration, preferably about approximately 30 minutes prior to administration.

### Administration

The composition for use as a vaccine according to the invention is administered in ovo during the final quarter of incubation of a domesticated bird. In an embodiment, the composition for use as a vaccine is administered in ovo from days 15 to 20 of incubation. In a further embodiment, the composition for use as a vaccine is administered in ovo from day 17 to 19.5 of incubation. In a further embodiment, the composition for use as a vaccine is administered in ovo from day 18 to 19.2. In a further embodiment, the composition for use as a vaccine is administered in ovo at approximately day 18.5 of incubation.

In an embodiment, the composition, the in ovo vaccine, or the composition for use as a vaccine is an aqueous suspension.

In an embodiment, the composition, the in ovo vaccine, or the composition for use as a vaccine comprises about between 100 and 6000 sporulated oocysts of E. acervulina per dose, preferably about between 200 and 5000 sporulated oocysts of E. acervulina per dose, more preferably about between 300 and 2000 sporulated oocysts of E. acervulina per dose, more preferably about between 400 and 1000 sporulated oocysts of E. acervulina per dose, and even more preferably about between 598 and 809 sporulated oocysts of E. acervulina per dose.

In a further embodiment, the composition, the in ovo vaccine, or the composition for use as a vaccine comprises about between 50 and 5000 sporulated oocysts of E. maxima per dose, preferably about between 100 and 4000 sporulated oocysts of E. maxima per dose, more preferably about between 150 and 3000 sporulated oocysts of E. maxima per dose, more preferably about between 200 and 1000 sporulated oocysts of E. maxima per dose, more preferably about between 300 and 600 sporulated oocysts of E. maxima per dose, and even more preferably about between 352 and 476 sporulated oocysts of E. maxima per dose.

In a further embodiment, the composition, the in ovo vaccine, or the composition for use as a vaccine comprises about between 50 and 3000 sporulated oocysts of E. praecox per dose, preferably about between 100 and 2000 sporulated oocysts of E. praecox per dose, more preferably about between 200 and 1000 sporulated oocysts of E. praecox per dose, more preferably about between 200 and 500 sporulated oocysts of E. praecox per dose, and even more preferably about between 235 and 317 sporulated oocysts of E. praecox per dose.

In a further embodiment, the composition, the in ovo vaccine, or the composition for use as a vaccine comprises about between 50 and 3000 sporulated oocysts of E. tenella per dose, preferably about between 100 and 2000 sporulated oocysts of E. tenella per dose, more preferably about between 200 and 1000 sporulated oocysts of E. tenella per dose, more preferably about between 200 and 500 sporulated oocysts of E. tenella per dose, and even more preferably about between 221 and 299 sporulated oocysts of E. tenella per dose.

In an embodiment, the amount of sporulated oocysts per dose for each Eimeria species are the same recited supra when the different combinations of Eimeria species of the invention are selected: E. acervulina, and E. maxima; E. acervulina, and E. praecox; E. acervulina, and E. tenella; E. acervulina, E. maxima, and E. praecox; E. acervulina, E. maxima, and E. tenella; and E. acervulina, E. praecox and E. tenella.

In a preferred embodiment, the composition, the in ovo vaccine, or the composition for use as a vaccine comprises about between 100 and 6000 sporulated oocysts of E. acervulina per dose, preferably about between 200 and 5000 sporulated oocysts of E. acervulina per dose, more preferably about between 300 and 2000 sporulated oocysts of E. acervulina per dose, more preferably about between 400 and 1000 sporulated oocysts of E. acervulina per dose, and even more preferably about between 598 and 809 sporulated oocysts of E. acervulina per dose; about between 50 and 5000 sporulated oocysts of E. maxima per dose, preferably about between 100 and 4000 sporulated oocysts of E. maxima per dose, more preferably about between 150 and 3000 sporulated oocysts of E. maxima per dose, more preferably about between 200 and 1000 sporulated oocysts of E. maxima per dose, more preferably about between 300 and 600 sporulated oocysts of E. maxima per dose, and even more preferably about between 352 and 476 sporulated oocysts of E. maxima per dose; about between 50 and 3000 sporulated oocysts of E. praecox per dose, preferably about between 100 and 2000 sporulated oocysts of E. praecox per dose, more preferably about between 200 and 1000 sporulated oocysts of E. praecox per dose, more preferably about between 200 and 500 sporulated oocysts of E. praecox per dose, and even more preferably about between 235 and 317 sporulated oocysts of E. praecox per dose; and about between 50 and 3000 sporulated oocysts of E. tenella per dose, preferably about between 100 and 2000 sporulated oocysts of E. tenella per dose, more preferably about between 200 and 1000 sporulated oocysts of E. tenella per dose, more preferably about between 200 and 500 sporulated oocysts of E. tenella per dose, and even more preferably about between 221 and 299 sporulated oocysts of E. tenella per dose.

In a preferred embodiment, the composition, the in ovo vaccine, or the composition for use as a vaccine comprises about between 598 and 809 sporulated oocysts of E. acervulina per dose, about between 352 and 476 sporulated oocysts of E. maxima per dose, about between 235 and 317 sporulated oocysts of E. praecox per dose, and about between 221 and 299 sporulated oocysts of E. tenella per dose.

In an embodiment of the composition or the in ovo vaccine of the invention, the amount of sporulated oocysts from E. acervulina is about between 200 and 5000 per dose, more preferably about between 300 and 2000 per dose, more preferably about between 400 and 1000 per dose, and even more preferably about between 598 and 809 per dose; the amount of sporulated oocysts from E. maxima is about between 100 and 4000 per dose, more preferably about between 150 and 3000 per dose, more preferably about between 200 and 1000 per dose, more preferably about between 300 and 600 per dose, and even more preferably about between 352 and 476 per dose; the amount of sporulated oocysts from E. praecox is about between 100 and 2000 per dose, more preferably about between 200 and 1000 per dose, more preferably about between 200 and 500 per dose, and even more preferably about between 235 and 317 per dose; and the amount of sporulated oocysts from E. tenella is about between 100 and 2000 per dose, more preferably about between 200 and 1000 per dose, more preferably about between 200 and 500 per dose, and even more preferably about between 221 and 299 per dose.

As shown in Example 7, it was observed an unexpected earlier (on day 3) and higher (70000 oocysts' output per gram fresh faeces) peak of oocysts' elimination profile for the in ovo live attenuated Eimeria spp. vaccine compositions of the invention compared to the coarse spray administration (on day 6, 40000 oocysts' output per gram fresh faeces), indicating that the in ovo vaccine composition of the invention is able to elicit an earlier immune response which in addition is protective against Eimeria spp. infections in vaccinated subjects.

Dose volumes are herein discussed in the general description of immunization and vaccination methods, and can also be determined by the skilled artisan from this disclosure read in conjunction with the common knowledge in the art, without any undue experimentation.

In an embodiment, the composition for use as a vaccine of the invention is further conveniently diluted in a vehicle prior to the in ovo administration. Said vehicle can be a diluent or solvent. Said diluent or solvent is an aqueous solution. The diluent or solvent may be selected from any physiological medium, preferably an aqueous solution comprising a salt selected from disodium phosphate, potassium dihydrogen phosphate, potassium chloride, sodium chloride, and mixtures thereof, in order to have the above content of oocysts per dose, or an aqueous solution comprising a sugar compound, such as sucrose or saccharose, a nitrogen source such as peptone (e.g., NZ amine) or a casein hydrolysate (e.g., Hy-case amino), in order to have the above content of oocysts per dose.

In an embodiment, the composition for use as a vaccine of the invention is diluted in a ratio of composition of the invention/diluent or solvent (v/v) of about 0.05/10, 0.1/10, 0.5/10, 1/10, 1.1/10, 1.2/10, 1.3/10, 1.31/10, 1.32/10, 1.33/10, 1.34/10, 1.35/10, 1.36/10, 1.37/10, 1.38/10, 1.39/10, 1.4/10, 1.5/10, 1.6/10, 1.7/10, 1.8/10, 1.9/10, 2/10, 3/10.

In an embodiment, the composition for use as a vaccine of the invention is diluted in a ratio of composition of the invention/diluent or solvent (v/v) of about 0.05/10, 0.1/10, 0.2/10, 1/10, 0.3/10, 0.4/10, 0.5/10, 0.6/10, 0.61/10, 0.62/10, 0.63/10, 0.64/10, 0.65/10, 0.66/10, 0.67/10, 0.68/10, 0.69/10, 0.7/10, 0.8/10, 0.9/10, 1/10, 2/10, 3/10.

In an embodiment, the composition for use as a vaccine of the invention is administered in a dose comprising between 0.050 ml and 0.20 ml.

In an embodiment, the composition for use as a vaccine of the invention is administered in a dose comprising between 0.050 ml and 0.10 ml.

In an embodiment, the composition for use as a vaccine of the invention is administered in a dose of 0.050 ml.

In an embodiment, the composition for use as a vaccine of the invention is administered in a dose of 0.10 ml.

Prevention and/or Treatment of an Eimeria Infection

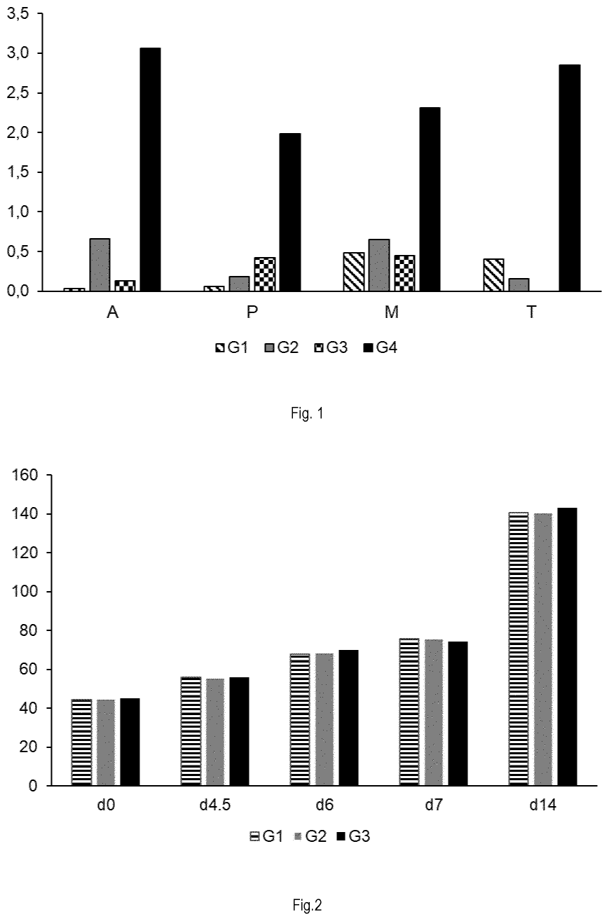

The present invention encompasses a composition for use as a vaccine in the prevention and/or treatment of an Eimeria infection. As shown in Example 2, it is clearly observed a significant reduction of the intestinal lesions in all the groups that received a vaccine comprising oocysts derived from precocious attenuated Eimeria strains when they are administered by in ovo administration, regardless of the oocyst dose of E. acervulina that they received, when compared to the birds of the control group (Group 4). This significant reduction is observed after the experimental infection (challenge) for all the Eimeria species (FIG. 1).

The composition for use as a vaccine of the invention comprising oocysts derived from precocious attenuated E. acervulina, and from at least one precocious attenuated strain selected from E. maxima, E. praecox and E. tenella is able to confer protection against a coccidia infection when it is administered by in ovo route.

The composition for use as a vaccine of the invention is suitable for the active immunization of embryonated eggs to prevent and/or reduce clinical signs, intestinal lesions and oocysts output associated with coccidiosis caused by Eimeria strains, preferably caused by any one of E. acervulina, E. maxima, E. praecox and E. tenella.

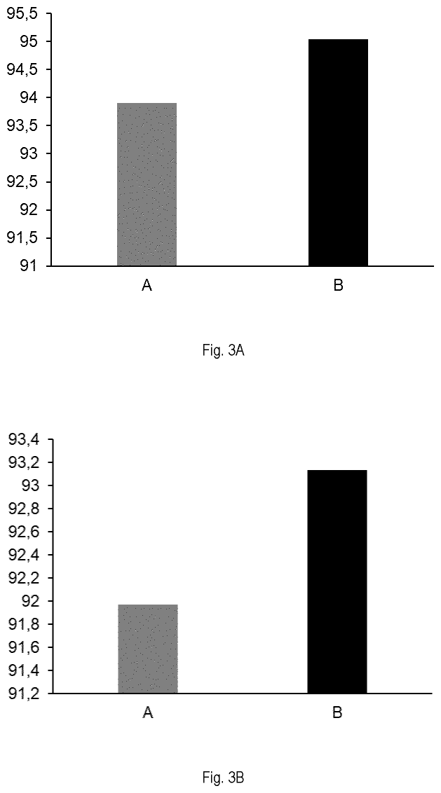

As shown in Example 3, it was unexpectedly found that the composition for use as a vaccine of the invention administered in ovo is safe, as no differences in the body weight are observed between animals from the vaccinated groups that received the composition of the invention, compared to animals from the control group. Furthermore, it was surprisingly found that animals that were inoculated with a dose of the composition of the invention ten times higher of the standard dose, had similar body weight than animals in the control group (FIG. 2). Therefore, these results confirm that a vaccine comprising oocysts derived from precocious attenuated strains of Eimeria is exceptionally safe when it is administered in ovo.

No alterations in the faeces' aspect and no general clinical signs were detected in said safety test. Thus, these results also confirm a good safety profile of the attenuated vaccine of the invention when it is administered in ovo.

Further, the administration of the composition of the invention to embryonated chicken eggs unexpectedly shows that the hatching rate is increased in the group vaccinated with the vaccine of the invention compared to the non-vaccinated control group, which received a mock-vaccine based exclusively on phosphate-buffered saline solution (PBS).

As shown in FIG. 3C, the overall mean of the hatchability rate is increased in 1.15% in animals that received the composition for use as a vaccine of the invention (Group B) compared to the control Group (Group A). Considering the large volume of eggs that are processed in industrialised and commercial hatcheries, this increase in the hatching rate percentage provides a surprising and important advantage over the current in ovo non-attenuated Eimeria vaccines.

In an embodiment of the present invention, the composition for use as a vaccine increases the hatchability rate of vaccinated embryonated chicken eggs compared to non-vaccinated embryonated chicken eggs that received a mock-vaccine, i.e., control group.

### Combination Vaccine

The composition, the in ovo vaccine, or the composition for use as a vaccine according to the invention can also include further active components such as a pathogen or immunogen, an antigen or an epitope of the pathogen thereof and at least one immunogen, antigen or epitope of another avian pathogen, avian parasite, avian bacteria or avian virus, i. e., the coccidiosis vaccine is combined with an additional avian vaccine composition.

In an embodiment, the composition, the in ovo vaccine, or the composition for use as a vaccine of the invention further comprises an additional avian vaccine, which comprises at least one immunogen, antigen or epitope of another avian pathogen.

Such an immunogen, antigen or epitope may be of bacterial, parasitic or viral origin and may be an inactivated or attenuated form of such bacteria, parasite or virus.

The additional avian vaccine comprises at least one immunogen, antigen or epitope of another avian pathogen, which may be without limitation the pathogen in inactivated or attenuated form. For avian multivalent immunogenic compositions, combination vaccines and multivalent vaccines, the additional avian pathogen (s), as to which additional avian antigen (s) or immunogen (s) or epitope (s) thereof are included in and/or expressed by the combination vaccine of the invention, are viruses, bacteria, or pathogens selected from the group of the Marek's disease virus (MDV) (e.g., serotypes 1 and 2, advantageously 1), Turkey Herpesvirus (HVT), Newcastle disease virus (NDV), paramyxoviruses other than Newcastle disease (PMV2 to PMV7), infectious bronchitis virus (IBV), infectious anaemia virus or chicken anaemia virus (CAV), infectious laryngotracheitis virus (ILTV), infectious bursal disease virus (IBDV, Gumboro disease), encephalomyelitis virus or avian encephalomyelitis virus (AEV or avian leukosis virus ALV), virus of haemorrhagic enteritis of turkeys (HEV), pneumovirosis virus (TRTV), fowl plague virus (avian influenza), chicken hydropericarditis virus, avian reoviruses, coccidia, egg drop syndrome virus (EDS), fowl pox, inclusion body hepatitis (adenovirus), lymphoproliferative disease virus (LDV), reticuloendotheliosis virus (REV), rotavirus enteritis, and turkey rhinotracheitis virus, Clostridium spp., Escherichia coli, Mycoplasma gallinarum, Mycoplasma gallisepticum, Haemophilus avium, Pasteurella gallinarum, Pasteurella multocida gallicida, Salmonella spp. and mixtures thereof.

In an embodiment, the additional avian vaccine comprises at least one immunogen, antigen or epitope of avian pathogen of an infectious bursal disease virus (IBDV, Gumboro disease).

Safety and efficacy data further demonstrate that the composition, the in ovo vaccine, or the composition for use as a vaccine of the invention can be mixed with an additional vaccine comprising at least one immunogen, antigen or epitope of another avian pathogen for the prevention and/or treatment of avian coccidiosis and at least for the prevention and/or treatment of another avian pathogen. The composition, the in ovo vaccine, or the composition for use as a vaccine of the invention can be combined, for example, with an additional vaccine comprising an infectious bursal disease virus (IBDV, Gumboro disease) antigen, prior to use and administered simultaneously by in ovo route. In an embodiment, the composition, the in ovo vaccine, or the composition of the invention is a combination vaccine for use in the prevention and/or treatment of avian coccidiosis and at least another avian pathogen by in ovo administration.

In an embodiment, the composition of the invention is a combination vaccine for use in the prevention and/or treatment of avian coccidiosis and Infectious Bursal Disease (Gumboro disease).

In an embodiment, the composition for use as a vaccine of the invention is for the prevention and/or treatment of an Eimeria infection and Infectious Bursal Disease virus (IBDV, Gumboro disease) infection. In an embodiment, the composition for use as a vaccine provides an immunogenic and/or protective immune response against an Eimeria infection and an IBDV infection.

In a further embodiment, the composition for use as a vaccine provides an immunogenic and/or protective immune response against an infection caused by at least one Eimeria species and against an IBDV.

In a further embodiment, the composition for use as a vaccine reduces or prevents hatched birds form one or more clinical signs associated with avian coccidiosis and Gumboro disease.

In an embodiment, the IBDV antigen is selected from inactivated IBDV, live attenuated IBDV, and antigenic subunit of IBDV. Preferably the IBDV antigen is a live attenuated IBDV, more preferably the IBDV antigen is an immune complex comprising live attenuated IBDV and IBDV-specific antibody solution.

In an embodiment, the additional avian vaccine comprises about between 10¹ and 10¹⁰ EID₅₀/ml (50% embryo infective dose/ml) of live attenuated IBDV, preferably about between 10² and 10⁹ EID₅₀/ml, more preferably about between 10³ and 10⁸ EID₅₀/ml, more preferably about between 10⁴ and 10⁷ EID₅₀/ml, and even more preferably about between 10⁵ and 10^{6.5} EID₅₀/ml of live attenuated IBDV.

In an embodiment, the additional avian vaccine comprising live attenuated IBDV further comprises an IBDV-specific antibody forming an immune complex of a IBDV antigen. Said IBDV-specific antibody has a titre that neutralizes at least 50% of the live attenuated IBDV. Preferably, the titre of the IBDV-specific antibody neutralizes at least 60%, more preferably at least 70%, more preferably at least 80%, more preferably at least 90%, more preferably at least 95%, more preferably at least 98%, and even more preferably at least 99% of the live attenuated IBDV. In a further embodiment, the IBDV-specific antibody has a titre that neutralizes the 100% of the live attenuated IBDV.

In an embodiment, the combination vaccine comprises about between 10¹ and 10¹⁰ EID₅₀/ml (50% embryo infective dose/ml) of live attenuated IBDV, preferably about between 10² and 10⁹ EID₅₀/ml, more preferably about between 10³ and 10⁸ EID₅₀/ml, more preferably about between 10⁴ and 10⁷ EID₅₀/ml, and even more preferably about between 10⁵ and 10^{6.5} EID₅₀/ml of live attenuated IBDV.

In an embodiment, the combination vaccine comprising live attenuated IBDV further comprises an IBDV-specific antibody forming an immune complex of a IBDV antigen. Said IBDV-specific antibody has a titre that neutralizes at least 50% of the live attenuated IBDV. Preferably, the titre of the IBDV-specific antibody neutralizes at least 60%, more preferably at least 70%, more preferably at least 80%, more preferably at least 90%, more preferably at least 95%, more preferably at least 98%, and even more preferably at least 99% of the live attenuated IBDV. In a further embodiment, the IBDV-specific antibody has a titre that neutralizes the 100% of the live attenuated IBDV.

Immune complex of a IBDV antigen comprising live attenuated IBDV and IBDV-specific antibody is known in prior art and commercially available. Said immune complex IBDV antigen may be obtained through commercial vaccine compositions, for example GUMBOHATCH® (Laboratorios HIPRA S.A., Avda. La Selva 135, 17170 Amer, Girona, Spain) or CEVAC TRANSUME® (Ceva Animal Health, S.A., 08028 Barcelona, Spain).

For mixed use, the onset and duration of immunity of the Eimeria species included in the composition for use as a combination vaccine of the invention are equivalent to those determined for said composition when used without the additional avian vaccine composition.

In an embodiment, the composition, the in ovo vaccine, or the composition for use as a vaccine of the invention and the additional avian vaccine are in separate containers, optionally in the same package, and optionally with instructions for admixture and/or administration.

In an embodiment, the composition, the in ovo vaccine, or the composition for use as a vaccine of the invention and the additional avian vaccine comprising IBDV antigen are in separate containers, optionally in the same package, and optionally with instructions for admixture and/or administration. In another embodiment, the composition, the in ovo vaccine, or the composition for use as a vaccine of the invention and the additional avian vaccine comprising IBDV antigen are in separate containers, optionally with instructions for admixture and/or administration.

In an embodiment, the composition for use as a vaccine of the invention is mixed with the additional avian vaccine prior to use and it is administered simultaneously in ovo to embryonated eggs of a domesticated bird.

In an embodiment, the composition for use as a vaccine of the invention is mixed with the additional avian vaccine prior to use and it is administered in ovo in a single dose.

In an embodiment, the composition for use as a vaccine of the invention is mixed with the additional avian vaccine comprising IBDV antigen prior to use and it is administered simultaneously in ovo to embryonated eggs of a domesticated bird.

In an embodiment, the composition for use as a vaccine of the invention is mixed with the additional avian vaccine comprising IBDV antigen prior to use and it is administered in ovo in a single dose.

In an embodiment, the composition for use as a vaccine of the invention and the additional avian vaccine are administered in ovo separately at substantially the same time.

In an embodiment, the composition for use as a vaccine of the invention and the additional avian vaccine comprising IBDV antigen are administered in ovo separately at substantially the same time.

In an embodiment, the composition for use as a vaccine of the invention and the additional avian vaccine comprising IBDV antigen are combined in a dose ratio selected from 2:1, 3:2, 1:1, 2:3, and 1:2 dose ratio referred to the vaccine of the invention:additional avian vaccine comprising IBDV. In a further embodiment the composition for use as a vaccine of the invention and the additional avian vaccine comprising IBDV antigen are combined in a 1:1 dose ratio.

A composition, or an in ovo vaccine, or an composition for use as a vaccine according to the invention that also comprises such an additional immunogenic component (additional immunogen, antigen or epitope) has the advantage that it induces an immune response or protection against several infections or diseases or causative agents thereof at the same time.

An aspect of the invention is a composition for use as a combination vaccine in the prevention and/or treatment of an Eimeria infection and Infectious Bursal Disease, wherein the composition comprises Eimeria oocysts from precocious attenuated strains of E. acervulina and from at least one precocious attenuated strain selected from E. maxima, E. praecox and E. tenella, wherein the vaccine is administered in ovo to embryonated eggs of a domesticated bird, in combination with an additional avian vaccine comprising at least one immunogen, antigen or epitope of avian pathogen of an infectious bursal disease virus (IBDV, Gumboro disease).

Another wording of this aspect of the invention is a composition for use as a combination vaccine in the prevention and/or treatment of an Eimeria infection and Infectious Bursal Disease, wherein the composition comprises Eimeria oocysts from precocious attenuated strains of E. acervulina and from at least one precocious attenuated strain selected from E. maxima, E. praecox and E. tenella, wherein an effective immunizing dose of the vaccine is administered in ovo to embryonated eggs of a domesticated bird, in combination with an additional avian vaccine comprising at least one immunogen, antigen or epitope of avian pathogen of an infectious bursal disease virus (IBDV, Gumboro disease).

In an embodiment, the Eimeria oocyst combination present in said composition for use as a combination vaccine consists of, or consists essentially of Eimeria oocysts from a precocious attenuated strain of E. acervulina, and from at least one precocious attenuated strain selected from E. maxima, E. praecox and E. tenella.

In an embodiment, the Eimeria oocyst combination present in said composition for use as a combination vaccine consists of, or consists essentially of Eimeria oocysts from precocious attenuated strains of E. acervulina, and E. maxima.

In an embodiment, the Eimeria oocyst combination present in said composition for use as a combination vaccine consists of, or consists essentially of Eimeria oocysts from precocious attenuated strains of E. acervulina, and E. praecox.

In an embodiment, the Eimeria oocyst combination present in said composition for use as a combination vaccine consists of, or consists essentially of Eimeria oocysts from precocious attenuated strains of E. acervulina, and E. tenella.

In a preferred embodiment, the Eimeria oocyst combination present in said composition for use as a combination vaccine comprises, consists of, or consists essentially of Eimeria oocysts from a precocious attenuated strain of E. acervulina, and from at least two precocious attenuated strains selected from E. maxima, E. praecox and E. tenella. In an embodiment, said composition for use as a combination vaccine consists of, or consists essentially of Eimeria oocysts from a precocious attenuated strain of E. acervulina, E. maxima, and E. praecox. In an embodiment, said composition for use as a combination vaccine consists of, or consists essentially of Eimeria oocysts from a precocious attenuated strain of E. acervulina, E. maxima, and E. tenella. In an embodiment, said composition consists of, or consists essentially of Eimeria oocysts from a precocious attenuated strain of E. acervulina, E. praecox and E. tenella.

In another preferred embodiment, the Eimeria oocyst combination present in said composition for use as a combination vaccine of the invention comprises, consists of, or consists essentially of a mixture of sporulated oocysts from precocious attenuated strains of E. acervulina, E. maxima, E. praecox, and E. tenella, i.e., in a preferred embodiment, the composition for use as a combination vaccine in the prevention and/or treatment of an Eimeria infection and Infectious Bursal Disease, comprises Eimeria oocysts from precocious attenuated strains selected from E. acervulina, E. maxima, E. praecox and E. tenella, wherein the vaccine is administered in ovo to embryonated eggs of a domesticated bird, in combination with an additional avian vaccine comprising at least one immunogen, antigen or epitope of avian pathogen of an infectious bursal disease virus (IBDV, Gumboro disease).

In another preferred embodiment, the Eimeria oocyst combination present in said composition for use as a combination vaccine of the invention comprises, consists of, or consists essentially of a mixture of sporulated oocysts from precocious attenuated strains of E. acervulina, E. maxima, E. praecox, and E. tenella, i.e., in a preferred embodiment, the composition for use as a combination vaccine in the prevention and/or treatment of an Eimeria infection and Infectious Bursal Disease, comprises Eimeria oocysts from precocious attenuated strains selected from E. acervulina, E. maxima, E. praecox and E. tenella, wherein an effective immunizing dose of the vaccine is administered in ovo to embryonated eggs of a domesticated bird, in combination with an additional avian vaccine comprising at least one immunogen, antigen or epitope of avian pathogen of an infectious bursal disease virus (IBDV, Gumboro disease).

Specific embodiments of the combination vaccine referring to the features of the E. acervulina precocious attenuated strain, the excipients included therein, the day and form of administration, the composition of sporulated oocysts, the volume of the dose of the vaccine, the number of doses, the IBDV antigen, and the ratio between the two vaccines are exposed in the above sections disclosing the features of the composition for use as a vaccine in the prevention and/or treatment of an Eimeria infection, and the features of the additional avian vaccine against the Infectious Bursal Disease (Gumboro disease).

In an embodiment, the additional vaccine comprising IBDV immunogen, antigen or epitope of avian pathogen of an infectious bursal disease virus (IBDV, Gumboro disease) is freeze-dried.

In an embodiment, the freeze-dried additional vaccine comprising IBDV immunogen, antigen or epitope of avian pathogen of an infectious bursal disease virus (IBDV, Gumboro disease) is diluted in a suitable vehicle, e.g., PBS, prior to the in ovo administration.

In an embodiment, the freeze-dried additional vaccine comprising IBDV immunogen, antigen or epitope of avian pathogen of an infectious bursal disease virus (IBDV, Gumboro disease) and the Eimeria vaccine are mixed in the same vehicle prior to the in ovo administration. The dose ratio of the combination vaccine of the invention has been described supra. In a particular embodiment, the combination vaccine of the invention is mixed in a 1:1 dose ratio.

The efficacy of the combination vaccine against Infectious Bursal Disease is effectively shown in Example 5. In said example, a combination of an attenuated Eimeria vaccine and an IBDV vaccine administered by in ovo route confirm the absence of detrimental effects on the efficacy of the IBDV vaccine, and also confirm that the combination vaccine is likewise efficacious against IBDV infection.

The efficacy of the combination vaccine against an Eimeria infection is effectively shown in Example 6. In said example, a combination of a live attenuated Eimeria vaccine against avian coccidiosis and a live attenuated vaccine against Infectious Bursal Disease was administered in ovo to embryonated chicken eggs against a challenge with four different Eimeria species, The results of the study demonstrate the efficacy of a combination vaccine of one dose of a live vaccine against avian Infectious Bursal Disease and a live attenuated vaccine against avian coccidiosis, when mixed prior to be administered in ovo to 18-day-old embryonated chicken eggs, against avian coccidiosis caused by Eimeria infections.

In an embodiment, the combination vaccine of the invention is for the immunization of embryonated eggs to prevent and/or reduce clinical signs associated with Gumboro disease or IBDV infections. In a particular embodiment, the combination vaccine of the invention prevents and/or reduces at least one of clinical signs associated with Gumboro disease or IBDV infection selected from the group consisting of mean acute histological functional score, macroscopic lesions, mononuclear infiltration oedema, inflammatory infiltrate, plical oedema, absence of serosal oedema and oedema in the muscular wall.

### Kit

In an embodiment the composition, the in ovo vaccine, or the composition for use as a vaccine of the invention is comprised in a kit.

In an embodiment, the composition, the in ovo vaccine, or the composition for use as a vaccine of the invention is comprised in a kit, wherein the kit comprises:
a) a container with a suspension of the Eimeria oocysts derived from precocious attenuated strains of coccidia in an aqueous medium comprising disodium phosphate, potassium dihydrogen phosphate, polysorbate, preferably polysorbate 80, potassium chloride, sodium chloride, and water, and
b) a container with an aqueous solution comprising disodium phosphate, potassium dihydrogen phosphate, potassium chloride, sodium chloride, and water.

In an embodiment, the kit or the kit for use of the vaccine composition of the invention further comprises a container comprising an additional vaccine comprising at least one immunogen, antigen or epitope of another avian pathogen against avian infections, as exposed above.

In another embodiment, the composition, the in ovo vaccine, or the composition for use as a vaccine of the invention is comprised in a kit, wherein, the kit comprises:
a) a container with a suspension of the Eimeria oocysts derived from precocious attenuated strains of coccidia in an aqueous medium comprising disodium phosphate, potassium dihydrogen phosphate, polysorbate, preferably polysorbate 80, potassium chloride, sodium chloride, and water, and
b) a container with an aqueous solution comprising disodium phosphate, potassium dihydrogen phosphate, potassium chloride, sodium chloride, and water, and
c) a container with additional vaccine comprising at least one immunogen or antigen of another avian pathogen against avian infections.

In another embodiment, the composition, the in ovo vaccine, or the composition for use as a vaccine of the invention is comprised in a kit, wherein the kit comprises:
a) a container with a suspension of the Eimeria oocysts derived from precocious attenuated strains of coccidia in an aqueous medium comprising disodium phosphate, potassium dihydrogen phosphate, polysorbate, preferably polysorbate 80, potassium chloride sodium chloride, and water, and
b) a container with an aqueous solution comprising disodium phosphate, potassium dihydrogen phosphate, potassium chloride, sodium chloride, and water, and
c) a container with an additional vaccine comprising at least one immunogen or antigen of IBDV (Gumboro disease) against avian infections.

In an embodiment, the kit can comprise one or multiple container or vials of the composition or the vaccine of the present invention, or one or multiple containers or vials of the composition or the vaccine of the present invention together with instructions for the administration to the birds at risk of Eimeria infections and/or at risk of Eimeria and IBDV infections (Gumboro disease).

Another aspect of the invention is a kit for use as a vaccine in the prevention and/or treatment of an Eimeria infection, wherein the vaccine is administered in ovo to embryonated eggs of a domesticated bird, and wherein the kit comprises the composition for use as a vaccine of the invention. Particularly, the kit comprises Eimeria oocysts from a precocious attenuated strain of E. acervulina, and from at least one precocious attenuated strain selected from E. maxima, E. praecox and E. tenella.

Another aspect of the invention is a kit for use as a vaccine comprising the composition for use as a vaccine of the invention. Preferably, the kit for use as a vaccine which comprises the composition for use as a vaccine comprises Eimeria oocysts from a precocious attenuated strain of E. acervulina, and from at least one precocious attenuated strain selected from E. maxima, E. praecox and E. tenella.

The invention comprises the following embodiments:

1.— A composition for use as a vaccine in the prevention and/or treatment of an Eimeria infection, wherein the composition comprises Eimeria oocysts from a precocious attenuated strain of E. acervulina, and from at least one precocious attenuated strain selected from E. maxima, E. praecox and E. tenella, wherein the vaccine is administered in ovo to embryonated eggs of a domesticated bird; preferably wherein an effective immunizing dose of the vaccine is administered in ovo to embryonated eggs of a domesticated bird.
2.— The composition for use as a vaccine according to embodiment 1, wherein said prevention and/or treatment results in an improvement in comparison to a non-treated control group of the same domesticated bird.
3.— The composition for use as a vaccine according to embodiment 1 or embodiment 2, wherein it provides an immunogenic and/or protective immune response against at least an Eimeria infection.
4.— The composition for use as a vaccine according to embodiment 1 or embodiment 2, wherein it provides an immunogenic and/or protective immune response against an infection caused by at least one Eimeria species.
5.— The composition for use as a vaccine according to any one of embodiments 1 to 4, wherein it reduces or prevents hatched birds from one or more clinical signs associated with avian coccidiosis.
6.— The composition for use as a vaccine according to embodiment 5, wherein the avian coccidiosis is caused by Eimeria infection.
7.— The composition for use as a vaccine according to embodiment 5 or embodiment 6, wherein the clinical signs are selected from the group consisting of intestinal lesions, decreased body weight, oocysts output, oocysts load, oocysts shedding, diarrhoea, dehydration, presence of blood in faeces, presence of mucous in faeces, Eimeria transmission, depression, ruffled feathers, arched body position, morbidity and mortality.
8.— The composition for use as a vaccine according to any one of embodiments 1 to 7, wherein it increases the hatching rate compared to a non-vaccinated control group.
9.— The composition for use as a vaccine according to embodiment 8, wherein the control group is being administered with PBS.
10.— The composition for use as a vaccine according to any one of embodiments 1 to 9, wherein the said composition reduces the oocysts output of hatched birds compared to hatched birds vaccinated in ovo with a composition comprising non-attenuated Eimeria strains.
11.— The composition for use as a vaccine according to any one of embodiments 1 to 10, wherein the domesticated bird is selected from chickens, turkeys, ducks, game birds and ratites; preferably, the domesticated birds are chicken.
12.— The composition for use as a vaccine according to any one of embodiments 1 to 11, wherein it is for the prevention of an Eimeria infection.
13.— The composition for use as a vaccine according to any one of embodiments 1 to 11, wherein it is for the treatment of an Eimeria infection.
14.— The composition for use as a vaccine according to any one of embodiments 1 to 11, wherein it is for the prevention of avian coccidiosis.
15.— The composition for use as a vaccine according to any one of embodiments 1 to 11, wherein it is for the treatment of avian coccidiosis.
16.— The composition for use as a vaccine according to any one of embodiments 1 to 15, wherein it consists of, or consists essentially of Eimeria oocysts from a precocious attenuated strain of E. acervulina, and from at least one precocious attenuated strain selected from E. maxima, E. praecox and E. tenella.
17.— The composition for use as a vaccine according to embodiment 16, wherein it consists of, or consists essentially of Eimeria oocysts from precocious attenuated strains of E. acervulina, and E. maxima.
18.— The composition for use as a vaccine according to embodiment 16, wherein it consists of, or consists essentially of Eimeria oocysts from precocious attenuated strains of E. acervulina, and E. praecox.
19.— The composition for use as a vaccine according to embodiment 16, wherein it consists of, or consists essentially of Eimeria oocysts from precocious attenuated strains of E. acervulina, and E. tenella.
20.— The composition for use as a vaccine according to any one of embodiments 1 to 15, wherein it comprises, consists of, or consists essentially of Eimeria oocysts from a precocious attenuated strain of E. acervulina, and from at least two precocious attenuated strains selected from E. maxima, E. praecox and E. tenella.
21.— The composition for use as a vaccine according to embodiment 20, wherein it consists of, or consists essentially of Eimeria oocysts from a precocious attenuated strain of E. acervulina, E. maxima, and E. praecox.
22.— The composition for use as a vaccine according to embodiment 20, wherein it consists of, or consists essentially of Eimeria oocysts from a precocious attenuated strain of E. acervulina, E. maxima, and E. tenella.
23.— The composition for use as a vaccine according to embodiment 20, wherein it consists of, or consists essentially of Eimeria oocysts from a precocious attenuated strain of E. acervulina, E. praecox and E. tenella.
24.— The composition for use as a vaccine according to any one of embodiments 1 to 15, wherein it comprises, consists of, or consists essentially of a mixture of sporulated oocysts from precocious attenuated strains of E. acervulina, E. maxima, E. praecox, and E. tenella.
25.— The composition for use as a vaccine according to any one of embodiments 1 to 24, wherein it comprises Eimeria oocysts selected from E. acervulina precocious attenuated strain with reduced pathogenicity and reduced oocysts output in comparison to a parent E. acervulina strain in a period of 14 days.
26.— The composition for use as a vaccine according to embodiment 25, wherein the E. acervulina precocious attenuated strain used therein, when administered to the host, the host excretes from about 30% to about 75% less, preferably from about 35% to about 65% less, more preferably from about 40% to about 60% less, and yet more preferably from about 45% to about 55% less of oocysts during a period of 14 days compared to the oocysts excreted by a host that received the parent E. acervulina strain.
27.— The composition for use as a vaccine according to any one of embodiments 1 to 24, wherein the E. acervulina precocious attenuated strain used therein, when administered to the host, the host excretes in a period of 14 days a similar number of oocysts as the oocysts excreted by a host that received the E. acervulina strain 120718P5 in a period of 14 days.
28.— The composition for use as a vaccine according to any one of embodiments 1 to 24, wherein the E. acervulina precocious attenuated strain used therein, when administered to the host, the host excretes in a period of 14 days about ±75% of the oocysts excreted by a host that received the E. acervulina strain 120718P5, preferably about ±70%, preferably about ±60%, preferably about ±50%, preferably about ±40%, preferably about ±30% of the oocysts excreted by a host that received the E. acervulina strain 120718P5, more preferably about ±25%, more preferably about ±20%, more preferably about ±15% of the oocysts excreted by a host that received the E. acervulina strain 120718P5, and yet more preferably about ±10% of the oocysts excreted by a host that received the E. acervulina strain 120718P5.
29.— The composition for use as a vaccine according to any one of embodiments 1 to 28, wherein the E. acervulina precocious strain used therein is attenuated by repeated passages of selection for precocious development from a parent strain; preferably after performing at least 3 passages, at least 5 passages, at least 6 passages, at least 7 passages, at least 8 passages, at least 9 passages, at least 10 passages, or at least 11 passages.
30.— The composition for use as a vaccine according to embodiment 29, wherein the E. acervulina precocious strain is attenuated after performing at least 5 passages of selection for precocious development from the parent strain.
31.— The composition for use as a vaccine according to any one of embodiments 1 to 30, wherein the E. acervulina precocious attenuated strain used therein is E. acervulina strain 120718P5, deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021.
32.— The composition for use as a vaccine according to any one of embodiments 1 to 31, wherein it comprises at least a pharmaceutically or veterinary acceptable carrier or vehicle or excipient or adjuvant.
33.— The composition for use as a vaccine according to embodiment 32, wherein the pharmaceutically or veterinary acceptable carrier or vehicle or excipients include wetting agents, dispersant agents, emulsifying agents, buffer agents, salts, stabilizing agents, proteins, water, saline, buffered saline, physiologically acceptable medium, and mixtures thereof.
34.— The composition for use as a vaccine according to embodiment 33, wherein the pharmaceutically or veterinary acceptable carrier or vehicle or excipient is selected from the group of sodium chloride, potassium chloride, a phosphate buffer, a surfactant, and mixtures thereof.
35.— The composition for use as a vaccine according to embodiment 34, wherein it comprises as vehicle and excipients a combination of disodium phosphate, potassium dihydrogen phosphate, polysorbate, potassium chloride, sodium chloride, and water.
36.— The composition for use as a vaccine according to embodiment 35, wherein it comprises polysorbate at a concentration ranging from about 0.001% to about 1% (v/v), from about 0.005% to about 0.5% (v/v), from about 0.006% to about 0.3% (v/v), from about 0.007% to about 0.1% (v/v), or from about 0.005% to about 0.05% (v/v).
37.— The composition for use as a vaccine according to any one of embodiments 33 to 36, wherein it comprises or consist essentially of one or more adjuvants selected from polymers of acrylic or methacrylic acid, maleic anhydride and alkenyl derivative polymers, immunostimulating sequences (ISS), an oil in water emulsion, cationic lipids containing a quaternary ammonium salt, cytokines, aluminium hydroxide, aluminium phosphate, and mixtures thereof.
38.— The composition for use as a vaccine according to any one of embodiments 1 to 37, wherein it is administered in ovo during the final quarter of incubation of a domesticated bird, preferably from days 15 to 20 of incubation, preferably from day 17 to 19.5 of incubation, preferably from day 18 to 19.2, and more preferably at approximately day 18.5 of incubation.
39.— The composition for use as a vaccine according to any one of embodiments 1 to 38, wherein it is an aqueous suspension.
40.— The composition for use as a vaccine according to any one of embodiments 1 to 39, wherein it comprises about between 100 and 6000 sporulated oocysts of E. acervulina per dose, preferably about between 200 and 5000 sporulated oocysts of E. acervulina per dose, more preferably about between 300 and 2000 sporulated oocysts of E. acervulina per dose, more preferably about between 400 and 1000 sporulated oocysts of E. acervulina per dose, and even more preferably about between 598 and 809 sporulated oocysts of E. acervulina per dose.
41.— The composition for use as a vaccine according to any one of embodiments 1 to 40, wherein it comprises about between 50 and 5000 sporulated oocysts of E. maxima per dose, preferably about between 100 and 4000 sporulated oocysts of E. maxima per dose, more preferably about between 150 and 3000 sporulated oocysts of E. maxima per dose, more preferably about between 200 and 1000 sporulated oocysts of E. maxima per dose, more preferably about between 300 and 600 sporulated oocysts of E. maxima per dose, and even more preferably about between 352 and 476 sporulated oocysts of E. maxima per dose.
42.— The composition for use as a vaccine according to any one of embodiments 1 to 41, wherein it comprises about between 50 and 3000 sporulated oocysts of E. praecox per dose, preferably about between 100 and 2000 sporulated oocysts of E. praecox per dose, more preferably about between 200 and 1000 sporulated oocysts of E. praecox per dose, more preferably about between 200 and 500 sporulated oocysts of E. praecox per dose, and even more preferably about between 235 and 317 sporulated oocysts of E. praecox per dose.
43.— The composition for use as a vaccine according to any one of embodiments 1 to 42, wherein it comprises about between 50 and 3000 sporulated oocysts of E. tenella per dose, preferably about between 100 and 2000 sporulated oocysts of E. tenella per dose, more preferably about between 200 and 1000 sporulated oocysts of E. tenella per dose, more preferably about between 200 and 500 sporulated oocysts of E. tenella per dose, and even more preferably about between 221 and 299 sporulated oocysts of E. tenella per dose.
44.— The composition for use as a vaccine according to any one of embodiments 1 to 43, wherein it comprises about between 598 and 809 sporulated oocysts of E. acervulina per dose, about between 352 and 476 sporulated oocysts of E. maxima per dose, about between 235 and 317 sporulated oocysts of E. praecox per dose, and about between 221 and 299 sporulated oocysts of E. tenella per dose.
45.— The composition for use as a vaccine according to any one of embodiments 1 to 44, wherein it is further diluted in a vehicle prior to the in ovo administration, which is an aqueous solution.
46.— The composition for use as a vaccine according to embodiment 45, wherein the aqueous solution comprises a) a salt selected from disodium phosphate, potassium dihydrogen phosphate, potassium chloride, sodium chloride, and mixtures thereof; b) a sugar compound; or c) a nitrogen source.
47.— The composition for use as a vaccine according to any one of embodiments 1 to 46, wherein it is administered in a dose comprising between 0.050 ml and 0.20 ml, preferably between 0.050 ml and 0.10 ml.
48.— The composition for use as a vaccine according to embodiment 47, wherein it is administered in a dose of 0.050 ml.
49.— The composition for use as a vaccine according to embodiment 47, wherein it is administered in a dose of 0.10 ml.
50.— The composition for use as a vaccine according to any one of embodiments 1 to 49, wherein it further comprises an additional avian vaccine, which comprises at least one immunogen, antigen or epitope of another avian pathogen.
51.— The composition for use as a vaccine according to embodiment 50, wherein the additional avian pathogen is selected from the group of the Marek's disease virus (MDV), Turkey Herpesvirus (HVT), Newcastle disease virus (NDV), paramyxoviruses other than Newcastle disease (PMV2 to PMV7), infectious bronchitis virus (IBV), infectious anaemia virus or chicken anaemia virus (CAV), infectious laryngotracheitis virus (ILTV), infectious bursal disease virus (IBDV, Gumboro disease), encephalomyelitis virus or avian encephalomyelitis virus (AEV or avian leukosis virus ALV), virus of haemorrhagic enteritis of turkeys (HEV), pneumovirosis virus (TRTV), fowl plague virus (avian influenza), chicken hydropericarditis virus, avian reoviruses, coccidia, egg drop syndrome virus (EDS), fowl pox, inclusion body hepatitis (adenovirus), lymphoproliferative disease virus (LDV), reticuloendotheliosis virus (REV), rotavirus enteritis, and turkey rhinotracheitis virus, Clostridium spp., Escherichia coli, Mycoplasma gallinarum, Mycoplasma gallisepticum, Haemophilus avium, Pasteurella gallinarum, Pasteurella multocida gallicida, Salmonella spp., and mixtures thereof.
52.— The composition for use as a vaccine according to embodiments 50 or 51, wherein the additional avian vaccine comprises at least one immunogen, antigen or epitope of avian pathogen of an infectious bursal disease virus (IBDV, Gumboro disease).
53.— The composition for use as a vaccine according to any one of embodiments 50 to 52, wherein it is for the prevention and/or treatment of an Eimeria infection and Infectious Bursal Disease virus (IBDV, Gumboro disease) infection; particularly, wherein it is for the prevention and/or treatment of avian coccidiosis and Infectious Bursal Disease (Gumboro disease).
54.— The composition for use as a vaccine according to embodiment 52, wherein it provides an immunogenic and/or protective immune response against an Eimeria infection and an IBDV infection.
55.— The composition for use as a vaccine according to embodiment 52, wherein it provides an immunogenic and/or protective immune response against an infection caused by at least one Eimeria species and against an IBDV.
56.— The composition for use as a vaccine according to embodiment 52, wherein it reduces or prevents hatched birds form one or more clinical signs associated with avian coccidiosis and Gumboro disease.
57.— The composition for use as a vaccine according to any one of embodiments 52 to 56, wherein the IBDV antigen is selected from inactivated IBDV, live attenuated IBDV, and antigenic subunit of IBDV, preferably the IBDV antigen is a live attenuated IBDV, more preferably the IBDV antigen is an immune complex comprising live attenuated IBDV and IBDV-specific antibody solution.
58.— The composition for use as a vaccine according to embodiment 57, wherein the additional avian vaccine comprises about between 10¹ and 10¹⁰ EID₅₀/ml (50% embryo infective dose/ml) of live attenuated IBDV, preferably about between 10² and 10⁹ EID₅₀/ml, more preferably about between 10³ and 10⁸ EID₅₀/ml, more preferably about between 10⁴ and 10⁷ EID₅₀/ml, and even more preferably about between 10⁵ and 10^{6.5} EID₅₀/ml of live attenuated IBDV.
59.— The composition for use as a vaccine according to any one of embodiments 50 to 58, wherein said composition and the additional avian vaccine are in separate containers, optionally with instructions for admixture and/or administration.
60.— The composition for use as a vaccine according to embodiment 59, wherein said composition is mixed with the additional avian vaccine prior to use and it is administered simultaneously in ovo to embryonated eggs of a domesticated bird.
61.— The composition for use as a vaccine according to embodiment 60, wherein said composition is administered in a single dose.
62.— The composition for use as a vaccine according to embodiment 59, wherein said composition and the additional avian vaccine are administered in ovo separately at substantially the same time.
63.— The composition for use as a vaccine according to any one of embodiments 1 to 62, wherein it is comprised in a kit.
64.— The composition for use as a vaccine according to embodiment 63, wherein the kit comprises:
a) a container with a suspension of the Eimeria oocysts derived from precocious attenuated strains of coccidia in an aqueous medium comprising disodium phosphate, potassium dihydrogen phosphate, polysorbate, preferably polysorbate 80, potassium chloride, sodium chloride, and water, and
b) a container with an aqueous solution comprising disodium phosphate, potassium dihydrogen phosphate, potassium chloride, sodium chloride, and water.
   65.— The composition for use as a vaccine or as a combination vaccine according to embodiment 63, wherein the kit comprises:
a) a container with a suspension of the Eimeria oocysts derived from precocious attenuated strains of coccidia in an aqueous medium comprising disodium phosphate, potassium dihydrogen phosphate, polysorbate, preferably polysorbate 80, potassium chloride, sodium chloride, and water, and
b) a container with an aqueous solution comprising disodium phosphate, potassium dihydrogen phosphate, potassium chloride, sodium chloride, and water, and
c) a container with additional vaccine comprising at least one immunogen or antigen of another avian pathogen against avian infections.
   66.— The composition for use as a vaccine or as a combination vaccine according to embodiment 63, wherein the kit comprises:
a) a container with a suspension of the Eimeria oocysts derived from precocious attenuated strains of coccidia in an aqueous medium comprising disodium phosphate, potassium dihydrogen phosphate, polysorbate, preferably polysorbate 80, potassium chloride sodium chloride, and water, and
b) a container with an aqueous solution comprising disodium phosphate, potassium dihydrogen phosphate, potassium chloride, sodium chloride, and water, and
c) a container with an additional vaccine comprising at least one immunogen or antigen of IBDV (Gumboro disease).
   67.— The composition for use as a vaccine according to any one of embodiments 63 to 66, wherein the kit comprises one or multiple container or vials of said vaccine, or one or multiple containers or vials of said vaccine together with instructions for the administration to the birds at risk of Eimeria infections and/or at risk of Eimeria and IBDV infections (Gumboro disease).
   68.— A composition for use as a combination vaccine in the prevention and/or treatment of an Eimeria infection and Infectious Bursal Disease, wherein the composition comprises Eimeria oocysts from precocious attenuated strains of E. acervulina and from at least one precocious attenuated strain selected from E. maxima, E. praecox and E. tenella, wherein the vaccine is administered in ovo to embryonated eggs of a domesticated bird, in combination with an additional avian vaccine comprising at least one immunogen, antigen or epitope of avian pathogen of an infectious bursal disease virus (IBDV, Gumboro disease).
   69.— The composition for use as a combination vaccine according to embodiment 68, wherein the Eimeria oocysts combination present in said composition is selected according to any one of embodiments 16 to 24.
   70.— The composition for use as a combination vaccine according to embodiment 68 or embodiment 69, wherein the additional vaccine comprising IBDV immunogen, antigen or epitope of avian pathogen of an infectious bursal disease virus (IBDV, Gumboro disease) is freeze-dried.
   71.— The composition for use as a combination vaccine according to embodiment 70, wherein the freeze-dried additional vaccine is diluted in a suitable vehicle prior to the in ovo administration.
   72.— The composition for use as a combination vaccine according to embodiment 71, wherein the freeze-dried additional vaccine comprising IBDV immunogen, antigen or epitope of avian pathogen of an infectious bursal disease virus (IBDV, Gumboro disease) and the Eimeria vaccine are mixed in the same vehicle prior to the in ovo administration.
   73.— The composition for use as a combination vaccine according to any one of embodiments 68 to 72, wherein it is for the immunization of embryonated eggs to prevent and/or reduce clinical signs associated with Gumboro disease or IBDV infections.
   74.— The composition for use as a combination vaccine according to embodiment 73, wherein it prevents and/or reduces at least one of clinical signs associated with Gumboro disease or IBDV infection selected from the group consisting of mean acute histological functional score, macroscopic lesions, mononuclear infiltration oedema, inflammatory infiltrate, plical oedema, absence of serosal oedema, and oedema in the muscular wall.
   75.— A kit for use as a vaccine comprising the composition for use as a vaccine according to any one of embodiments 1 to 62.
   76.— A kit for use as a vaccine in the prevention and/or treatment of an Eimeria infection, wherein the vaccine is administered in ovo to embryonated eggs of a domesticated bird, and wherein the kit comprises the composition for use as a vaccine according to any one of embodiments 1 to 62.
   77.— A composition comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose.
   78.— A composition comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose; with the proviso that the composition does not comprise E. mitis sporulated oocysts.
   79.— A composition comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose, and wherein the composition is suitable for preparing an in ovo vaccine.
   80.— A composition comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, the amount of sporulated oocysts from E. maxima is about between 50 and 5000 per dose, the amount of sporulated oocysts from E. praecox is about between 50 and 3000 per dose, and the amount of sporulated oocysts from E. tenella is about between 50 and 3000 per dose, and wherein the composition is suitable for preparing an in ovo vaccine; with the proviso that the composition does not comprise E. mitis sporulated oocysts.
   81.— The composition according to any one of embodiments 77 to 80 which is an immunogenic composition.
   82.— The composition according to any one of embodiments 77 to 81, which comprises, consists of, or consists essentially of, sporulated oocysts from precocious attenuated strains of E. acervulina, E. maxima, E. praecox, and E. tenella.
   83.— The composition according to any one of embodiments 77 to 81, which comprises, consists of, or consists essentially of, sporulated oocysts from precocious attenuated strains of E. acervulina and E. maxima; E. acervulina and E. praecox; E. acervulina and E. tenella; E. acervulina and from at least two precocious attenuated strains selected from E. maxima, E. praecox and E. tenella; E. acervulina, E. maxima, and E. praecox; E. acervulina, E. maxima, and E. tenella; or E. acervulina, E. praecox and E. tenella 84.— The composition according to any one of embodiments 77 to 83, wherein it comprises Eimeria oocysts selected from E. acervulina precocious attenuated strain with reduced pathogenicity and reduced oocysts output in comparison to a parent E. acervulina strain in a period of 14 days.
   85.— The composition according to any one of embodiments 77 to 84, wherein the E. acervulina precocious attenuated strain, when administered to the host, the host excretes from about 30% to about 75% less, preferably from about 35% to about 65% less, more preferably from about 40% to about 60% less, and yet more preferably from about 45% to about 55% less of oocysts during a period of 14 days compared to the oocysts excreted by a host that received the parent E. acervulina strain.
   86.— The composition according to any one of embodiments 77 to 85, wherein the E. acervulina precocious attenuated strain, when administered to the host, the host excretes in a period of 14 days a similar number of oocysts as the oocysts excreted by a host that received the E. acervulina strain 120718P5 in a period of 14 days.
   87.— The composition according to any one of embodiments 77 to 86, wherein the E. acervulina precocious attenuated strain, when administered to the host, the host excretes in a period of 14 days about ±75% of the oocysts excreted by a host that received the E. acervulina strain 120718P5, preferably about ±70%, preferably about ±60%, preferably about ±50%, preferably about ±40%, preferably about ±30% of the oocysts excreted by a host that received the E. acervulina strain 120718P5, more preferably about ±25%, more preferably about ±20%, more preferably about ±15% of the oocysts excreted by a host that received the E. acervulina strain 120718P5, and yet more preferably about ±10% of the oocysts excreted by a host that received the E. acervulina strain 120718P5.
   88.— The composition according to any one of embodiments 77 to 87, wherein the E. acervulina precocious strain is attenuated by repeated passages of selection for precocious development from a parent strain; preferably after performing at least 3 passages, at least 5 passages, at least 6 passages, at least 7 passages, at least 8 passages, at least 9 passages, at least 10 passages, or at least 11 passages.
   89.— The composition according to embodiment 88, wherein the E. acervulina precocious strain is attenuated after performing at least 5 passages of selection for precocious development from the parent strain.
   90.— The composition according to any one of embodiments 77 to 89, wherein the E. acervulina precocious attenuated strain is E. acervulina strain 120718P5, deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021.
   91.— The composition according to any one of embodiments 77 to 90, wherein the amount of sporulated oocysts from E. acervulina is about between 200 and 5000 per dose, more preferably about between 300 and 2000 per dose, more preferably about between 400 and 1000 per dose, and even more preferably about between 598 and 809 per dose.
   92.— The composition according to any one of embodiments 77 to 91, wherein the amount of sporulated oocysts from E. maxima is about between 100 and 4000 per dose, more preferably about between 150 and 3000 per dose, more preferably about between 200 and 1000 per dose, more preferably about between 300 and 600 per dose, and even more preferably about between 352 and 476 per dose.
   93.— The composition according to any one of embodiments 77 to 92, wherein the amount of sporulated oocysts from E. praecox is about between 100 and 2000 per dose, more preferably about between 200 and 1000 per dose, more preferably about between 200 and 500 per dose, and even more preferably about between 235 and 317 per dose.
   94.— The composition according to any one of embodiments 77 to 93, wherein the amount of sporulated oocysts from E. tenella is about between 100 and 2000 per dose, more preferably about between 200 and 1000 per dose, more preferably about between 200 and 500 per dose, and even more preferably about between 221 and 299 per dose.
   95.— The composition according to any one of embodiments 77 to 94, wherein it comprises about between 598 and 809 sporulated oocysts of E. acervulina per dose, about between 352 and 476 sporulated oocysts of E. maxima per dose, about between 235 and 317 sporulated oocysts of E. praecox per dose, and about between 221 and 299 sporulated oocysts of E. tenella per dose.
   96.— An in ovo vaccine comprising the composition as defined in any one of embodiments 77 to 95.
   97.— An in ovo vaccine comprising the composition as defined in any one of embodiments 77 to 95; with the proviso that the in ovo vaccine does not comprise E. mitis sporulated oocysts.
   98. The in ovo vaccine according to embodiment 96 or 97 further comprising at least a pharmaceutically or veterinary acceptable carrier or vehicle or excipient or adjuvant.
   99.— The in ovo vaccine according to embodiment 98 wherein the at least a pharmaceutically or veterinary acceptable carrier or vehicle or excipient or adjuvant is suitable for use via in ovo.
   100.— The in ovo vaccine according to any one of embodiments 96 to 99, wherein it further comprises an additional avian vaccine, which comprises at least one immunogen, antigen or epitope of another avian pathogen.
   101.— The in ovo vaccine according to embodiment 100, wherein the additional avian pathogen is selected from the group of the Marek's disease virus (MDV), Turkey Herpesvirus (HVT), Newcastle disease virus (NDV), paramyxoviruses other than Newcastle disease (PMV2 to PMV7), infectious bronchitis virus (IBV), infectious anaemia virus or chicken anaemia virus (CAV), infectious laryngotracheitis virus (ILTV), infectious bursal disease virus (IBDV, Gumboro disease), encephalomyelitis virus or avian encephalomyelitis virus (AEV or avian leukosis virus ALV), virus of haemorrhagic enteritis of turkeys (HEV), pneumovirosis virus (TRTV), fowl plague virus (avian influenza), chicken hydropericarditis virus, avian reoviruses, coccidia, egg drop syndrome virus (EDS), fowl pox, inclusion body hepatitis (adenovirus), lymphoproliferative disease virus (LDV), reticuloendotheliosis virus (REV), rotavirus enteritis, and turkey rhinotracheitis virus, Clostridium spp., Escherichia coli, Mycoplasma gallinarum, Mycoplasma gallisepticum, Haemophilus avium, Pasteurella gallinarum, Pasteurella multocida gallicida, Salmonella spp., and mixtures thereof.
   102.— The in ovo vaccine according to embodiment 101, wherein the additional avian vaccine comprises at least one immunogen, antigen or epitope of avian pathogen of an infectious bursal disease virus (IBDV, Gumboro disease).
   103.— The in ovo vaccine according to embodiment 102, wherein the IBDV antigen is selected from inactivated IBDV, live attenuated IBDV, and antigenic subunit of IBDV, preferably the IBDV antigen is a live attenuated IBDV, more preferably the IBDV antigen is an immune complex comprising live attenuated IBDV and IBDV-specific antibody solution.
   104.— The in ovo vaccine according to embodiment 103, wherein the additional avian vaccine comprises about between 10¹ and 10¹⁰ EID₅₀/ml (50% embryo infective dose/ml) of live attenuated IBDV, preferably about between 10² and 10⁹ EID₅₀/ml, more preferably about between 10³ and 10⁸ EID₅₀/ml, more preferably about between 10⁴ and 10⁷ EID₅₀/ml, and even more preferably about between 10⁵ and 10^{6.5} EID₅₀/ml of live attenuated IBDV.
   105— The in ovo vaccine according to any one of embodiments 100 to 104, wherein said composition and the additional avian vaccine are in separate containers, optionally with instructions for admixture and/or administration.
   106.— The in ovo vaccine according to any one of embodiments 98 to 105, wherein it comprises as vehicle and excipients a combination of disodium phosphate, potassium dihydrogen phosphate, polysorbate, potassium chloride, sodium chloride, and water.
   107.— The in ovo vaccine according to any one of embodiments 98 to 106, wherein it comprises polysorbate at a concentration ranging from about 0.001% to about 1% (v/v), from about 0.005% to about 0.5% (v/v), from about 0.006% to about 0.3% (v/v), from about 0.007% to about 0.1% (v/v), or from about 0.005% to about 0.05% (v/v).
   108.— The in ovo vaccine according to any one of embodiments 98 to 107, wherein it comprises or consist essentially of one or more adjuvants selected from polymers of acrylic or methacrylic acid, maleic anhydride and alkenyl derivative polymers, immunostimulating sequences (ISS), an oil in water emulsion, cationic lipids containing a quaternary ammonium salt, cytokines, aluminium hydroxide, aluminium phosphate, and mixtures thereof.
   109.— A kit comprising the composition according to any one of embodiments 77 to 95 or the in ovo vaccine according to any one of embodiments 96 to 108.

In the following examples, different embodiments of the invention are provided for illustrative purposes which should be understood as non-limiting.

### EXAMPLES

### Example 1: Obtention of E. acervulina Strain 120718P5

The parent strain of the attenuated E. acervulina strain 120718P5 was isolated from a clinical case of diagnosed avian coccidiosis by scrapings of the duodenum of chicks of 30 days on a poultry farm in Spain in January 2017.

The chicks presented mild lesions characteristic of E. acervulina infection in the duodenum as described by Johnson et al., Anticoccidial drugs: lesion scoring technics in battery and floor-pen experiments with chickens, Exp. Parasitol., 1970, 28: 30-36.

Immature oocysts were yielded directly from scratches of the duodenal mucosa, homogenized together with 2.5% potassium dichromate and sporulated at 28.5° C. for 48 hours (suspension 1).

Sporulated oocysts of suspension 1 were then inoculated into 5 “coccidia-free” SPF chicks, in order to perform a multiplication passage. Faeces were recovered from 4 to 6 days post inoculation. All the birds survived to the different passages. At the end of the serial passages the animals were necropsied. At necropsy, the birds presented mild E. acervulina lesions in the duodenum. No other macroscopic lesions were detected all along the intestine or in other tissues. Oocysts were homogenized in 2.5% potassium dichromate solution, and subsequently sporulated at 28.5° C. for 48 hours (suspension 2).

To avoid any contamination by another Eimeria species affecting the domestic fowl, a population of oocysts was obtained from a single oocyst of the suspension 2. The chosen oocyst was isolated from the rest of oocysts present in the suspension 2 using a 0.1 ml microcapillary pipette with an aspirator tube. Only one oocyst was inoculated to each bird (3 weeks-old).

Oocysts derived from the oocyst inoculated birds were recovered from the faeces of one “coccidia-free” SPF chick and sporulated (suspension 3). Due to the small number of oocysts recovered, these oocysts were inoculated into another group of 5 “coccidia-free” SPF chicks, in order to obtain a larger number of oocysts (suspension 4).

The suspension 4 of sporulated oocysts obtained from this final multiplication passage was referenced as E. acervulina 120718 parent strain.

After that, E. acervulina 120718 parent strain was subsequently attenuated for precocity after performing laboratory passages in “coccidia-free” SPF chicks. Parent 120718 E. acervulina strain was submitted to 5 passages of selection for precocious development. The final strain obtained after passage 5 was referenced as E. acervulina 120718P5.

E. acervulina strain 120718P5 was deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021.

This strain 120718P5 showed a reduced pathogenicity and when administered to the host, the host excretes about 50% less of live oocysts compared to a host that received the parent E. acervulina strain during a period of 14 days.

### Example 2: Efficacy Study of a Live Vaccine Containing Precocious Attenuated Eimeria Oocysts by in Ovo Administration

A total of 160 embryonated poultry eggs were chosen for this study. Embryonated eggs were randomly assigned into 4 groups of 40 eggs each one. At day 18 of incubation embryonated eggs were injected by in ovo route with 0.1 ml of different experimental vaccines compositions according to the group assignment:
Group 1 (E. acervulina high dose): This group received a vaccine composition comprising 0.006 ml of 978 sporulated oocysts derived from attenuated E. acervulina strain 120718P5, 414 sporulated oocysts derived from attenuated E. maxima strain 013, 276 sporulated oocysts derived from attenuated E. praecox strain 007 and 260 sporulated oocysts derived from attenuated E. tenella strain 004 in phosphate-buffered saline solution (PBS) with 0.01% (v/v) of polysorbate 80, diluted in 0.094 ml of PBS. A total volume of 0.1 ml of the vaccine composition was injected to each egg.
Group 2 (E. acervulina mid dose): This group received a vaccine composition comprising 0.006 ml of 782 sporulated oocysts derived from attenuated E. acervulina strain 120718P5, 414 sporulated oocysts derived from attenuated E. maxima strain 013, 276 sporulated oocysts derived from attenuated E. praecox strain 007 and 260 sporulated oocysts derived from attenuated E. tenella strain 004 in phosphate-buffered saline solution (PBS) with 0.01% (v/v) of polysorbate 80, diluted in 0.094 ml of PBS. A total volume of 0.1 ml of the vaccine composition was injected to each egg.
Group 3 (E. acervulina low dose): This group received a vaccine composition comprising 0.006 ml of 587 sporulated oocysts derived from attenuated E. acervulina strain 120718P5, 414 sporulated oocysts derived from attenuated E. maxima strain 013, 276 sporulated oocysts derived from attenuated E. praecox strain 007 and 260 sporulated oocysts derived from attenuated E. tenella strain 004 in phosphate-buffered saline solution (PBS) with 0.01% (v/v) of polysorbate 80, diluted in 0.094 ml of PBS. A total volume of 0.1 ml of the vaccine composition was injected to each egg.
Group 4 (control group): This group was used as a positive control group. The birds received a mock-vaccine comprising only PBS.

All the oocysts comprised in the vaccine compositions derived from precocious attenuated Eimeria strains. The precocious attenuated strain of E. acervulina used in this study was the one obtained in Example 1. The precocious attenuated strains of E. maxima, E. praecox, E. tenella used in the vaccine compositions of the study were obtained from the commercial vaccine EVANT® (Laboratorios HIPRA, S.A., Avda. La Selva, 135, 17170 Amer, Girona, Spain), which is available on the European Union through the marketing authorisation issued by the EMA (EMEA/V/C/004902) on May 2, 2019. It is also available in other non-European countries through the corresponding national licenses.

21 days after hatch, 10 birds per group were randomly assigned into 4 different groups. Each group was infected (challenged) experimentally with different Eimeria species to assess the efficacy of the attenuated vaccine compositions administered at different doses of sporulated Eimeria oocysts when it was administered by in ovo route. Birds were experimentally infected by oral gavage administering 1 ml/bird of a preparation comprising oocysts from a virulent Eimeria strain, using an insulin syringe without needle. A first group was challenged with a preparation comprising 250.000 oocysts/ml derived from the virulent strain E. acervulina Houghton strain. A second group was challenged with a preparation comprising 100.000 oocysts/ml derived from the virulent strain E. maxima Weybridge strain. A third group was challenged with a preparation comprising 1.000.000 oocysts/ml derived from the virulent strain E. praecox Houghton strain. The fourth group was challenged with a preparation comprising 30.000 oocysts/ml derived from the virulent strain E. tenella Houghton strain. The distribution is described in Table I:

TABLE I

| | No. embryonated | | |
|---|---|---|---|
| Treatment Group | eggs | No. hatched birds | Challenge |
| 1 | 40 | 10 | E. acervulina |
| | | 10 | E. maxima |
| | | 10 | E. praecox |
| | | 10 | E. tenella |
| 2 | 40 | 10 | E. acervulina |
| | | 10 | E. maxima |
| | | 10 | E. praecox |
| | | 10 | E. tenella |
| 3 | 40 | 10 | E. acervulina |
| | | 10 | E. maxima |
| | | 10 | E. praecox |
| | | 10 | E. tenella |
| 4 | 40 | 10 | E. acervulina |
| | | 10 | E. maxima |
| | | 10 | E. praecox |
| | | 10 | E. tenella |

To assess the efficacy of the E. praecox strain 007 present in the vaccine composition administered by in ovo route, birds were ethically euthanized on day 4.5 after receiving the experimental infection with the E. praecox strain Houghton strain, and intestinal lesions post-challenge were determined.

To assess the efficacy of the E. acervulina strain 120718P5 and E. maxima strain 013 present in the vaccine composition administered by in ovo route, birds were ethically euthanized on day 6 after the experimental infection with E. acervulina strain Houghton strain and E. maxima strain Weybridge strain, and intestinal lesions post-challenge were determined.

To assess the efficacy of the E. tenella strain 004 present in the vaccine composition administered by in ovo route, birds were ethically euthanized on day 7 after the experimental infection with E. tenella strain Houghton strain, and intestinal lesions post-challenge were determined.

Intestinal lesions were evaluated following Johnson et al. op.cit., for E. acervulina, E. maxima and E. tenella. For E. praecox, the evaluation was performed according to the disclosure of Williams et al., Pathogenesis of Eimeria praecox in chickens: virulence of field strains compared with laboratory strains of E. praecox and Eimeria acervulina, Avian Pathol., 2009, 38(5), 359-366.

The mean of the intestinal lesion score per vaccine group and experimental infection group assignment was then calculated.

As shown in FIG. 1, it was clearly observed a significant reduction of the intestinal lesions in all the groups that received the vaccine compositions comprising oocysts derived from precocious attenuated Eimeria strains by in ovo administration, regardless of the oocyst dose of E. acervulina that they received (high, medium or low), when compared to the birds of the non-vaccinated control group (Group 4). This significant reduction was observed after the experimental infection (challenge) for all the Eimeria species.

Therefore, a vaccine comprising oocysts derived from precocious attenuated strains selected from E. acervulina, E. maxima, E. praecox and E. tenella is safe and able to generate an effective immune response against an Eimeria infection. The results show that the vaccine composition confers protection against avian coccidiosis when it is administered by in ovo route.

### Example 3: Safety Study of a Live Vaccine Containing Precocious Attenuated Eimeria Oocysts Administered by in Ovo Route

This was a 14-day study involving two treatments and one control group to evaluate the safety of one dose (1×) and an overdose (10×) of an attenuated Eimeria oocysts vaccine composition when it is administered in ovo to embryonated chicken eggs.

150 embryonated chicken eggs were chosen for the study. The eggs were randomly assigned into 3 different groups of 50 eggs each one. At day 18 of incubation the eggs were inoculated by in-ovo administration with a single dose (Group 1) and an overdose of ten times (10×) (Group 2) of an experimental attenuated vaccine against coccidiosis. Group 3 was left non-vaccinated and used as a control group.

Embryonated eggs in Group 1 (1×) received a 0.1 ml vaccine composition comprising 0.0069 ml of 703 sporulated oocysts derived from attenuated E. acervulina strain 120718P5, 414 sporulated oocysts derived from attenuated E. maxima strain 013, 276 sporulated oocysts derived from attenuated E. praecox strain 007 and 260 sporulated oocysts derived from attenuated E. tenella strain 004 in phosphate-buffered saline solution (PBS) with 0.01% (v/v) of polysorbate 80, diluted in 0.0931 ml of PBS. A total volume of 0.1 ml of the vaccine composition was injected to each egg.

Embryonated eggs in Group 2 (10×) received a 0.1 ml vaccine composition comprising 0.069 ml of 7030 sporulated oocysts derived from attenuated E. acervulina strain 120718P5, 4140 sporulated oocysts derived from attenuated E. maxima strain 013, 2760 sporulated oocysts derived from attenuated E. praecox strain 007 and 2600 sporulated oocysts derived from attenuated E. tenella strain 004 in phosphate-buffered saline solution (PBS) with 0.01% (v/v) of polysorbate 80, diluted in 0.031 ml of PBS. A total volume of 0.1 ml of the vaccine composition was injected to each egg.

Embryonated eggs in Group 3 received a 0.1 ml composition comprising a mock-vaccine based on phosphate-buffered saline solution (PBS) exclusively.

All the oocysts comprised in the vaccine formulations derived from precocious attenuated Eimeria strains. The precocious attenuated strain of E. acervulina used in this study was the one obtained in Example 1. The precocious attenuated strains of E. maxima, E. praecox, E. tenella used in the vaccine compositions of the study were obtained from the commercial vaccine EVANT® (Laboratorios HIPRA, S.A., Avda. La Selva, 135, 17170 Amer, Girona, Spain), which is available on the European Union through the marketing authorisation issued by the EMA (EMEA/V/C/004902) on May 2, 2019. It is also available in other non-European countries through the corresponding national licenses.

To determine the safety of the vaccine compositions, the body weight of birds was recorded on days 0, 4.5, 6, 7 and 14 after hatch.

The study revealed that there were no differences in the body weight between animals from the vaccinated groups, Group 1 and 2, compared to animals from the control group (Group 3). Surprisingly, animals in Group 2, that were inoculated with a oocysts dose ten times higher of the standard dose, had similar body weight than animals in the control group, Group 3 (FIG. 2). Therefore, these results confirm that a vaccine comprising oocysts derived from precocious attenuated strains of Eimeria is safe when it is administered in ovo even at doses above the recommended standard dose.

Clinical sings were also monitored during the study. No alterations in the faeces' aspect and no general clinical signs were detected in the vaccinated groups. Thus, these results also confirm an optimal safety profile of the attenuated vaccine when it is administered in ovo.

### Example 4: Hatchability Rate Study of a Live Vaccine Containing Precocious Attenuated Eimeria Oocysts by in Ovo Administration

As disclosed in the prior art in ovo vaccination may affect negatively the hatchability of embryonated eggs. This may be due to the type of vaccine administered to the embryonated eggs. For instance, the in ovo vaccination with Inovocox EM1 vaccine, which is a vaccine comprising live sporulated oocysts derived from non-attenuated Eimeria strains, reduced the hatchability rate between 1 to 2.59% in comparison with the diluent injected group (Sokale et al., 2017, op. cit., and Sokale et al., 2021, op. cit.).

This study assesses the safety in terms of hatchability rate of a vaccine comprising sporulated oocysts derived from precocious attenuated Eimeria strains, when it is administered by in ovo route, under field conditions.

Two commercial hatcheries were included in the study. The embryonated eggs from a homogenous batch (hens from same or similar age flocks) in each hatchery were distributed in two groups (Groups A and B). Each Group received a different vaccine as described below. All the vaccinations were performed in the hatchery after random distribution of the eggs in the two groups. The assignment of the vaccines to each group was done randomly in the hatchery. The embryonated eggs were injected by in ovo administration with 0.05 ml/egg of the vaccine at day 18 of incubation.

Group A: This group was used as the positive control group. The animals in this group received a mock-vaccine based on phosphate-buffered saline solution (PBS).

Group B: Animals in this group received a vaccine comprising 0.006 ml of a mixture comprising between 598-809 sporulated oocysts derived from attenuated E. acervulina strain 120718, between 352-476 sporulated oocysts derived from attenuated E. maxima strain 013, between 235-317 sporulated oocysts derived from attenuated E. praecox strain 007 and between 221-299 sporulated oocysts derived from attenuated E. tenella strain 004 in phosphate-buffered saline solution (PBS) with 0.01% (v/v) of polysorbate 80, diluted in 0.044 ml of diluent PBS. A total volume of 0.05 ml of the vaccine composition was injected to each egg.

All the oocysts comprised in the vaccine formulation derived from precocious attenuated Eimeria strains. The precocious attenuated strain of E. acervulina used in this study was the one obtained in Example 1. The precocious attenuated strains of E. maxima, E. praecox, E. tenella used in the vaccine compositions of the study were obtained from the commercial vaccine EVANT® (Laboratorios HIPRA, S.A., Avda. La Selva, 135, 17170 Amer, Girona, Spain), which is available on the European Union through the marketing authorisation issued by the EMA (EMEA/V/C/004902) on May 2, 2019. It is also available in other non-European countries through the corresponding national licenses.

The total number of embryonated eggs per each group varied depending on the hatchery. For the first hatchery (hatchery A) a total of 219,996 embryonated eggs were included in the study, 108,471 embryonated eggs were assigned to Group B, and 111,525 embryonated eggs were assigned to Group A (control group). For the second hatchery (hatchery B) a total of 144,476 embryonated eggs were included in the study, 72,187 embryonated eggs were assigned to Group B, and 72,289 embryonated eggs were assigned to Group A (control group).

The hatching rate was calculated as the number of eggs hatched out divided by the total number of fertile eggs in each group. For each batch of eggs and chickens included in the study, the hatching rate was compared between groups. Furthermore, the overall mean hatching rate obtained in each group among all the batches of eggs and chickens included in the study was compared between groups.

The results unexpectedly show that the hatching rate is increased in the group vaccinated with the vaccine of the invention (Group B) compared to the control group (Group A) in the two different hatcheries tested (FIGS. 3A and 3B). The overall mean of the hatchability rate is increased in 1.15% compared to the control Group (FIG. 3C). Considering the large volume of eggs that are processed in commercial hatcheries, this increase observed in the hatching rate percentage results in a surprising and an important advantage over the current in ovo non-attenuated Eimeria vaccines.

### Example 5: Efficacy of One Dose of the Combination of a Live Attenuated Eimeria Vaccine Against Avian Coccidiosis and a Live Attenuated Vaccine Against Infectious Bursal Disease (IBD) by in Ovo Administration to Embryonated Chicken Eggs Against a Challenge with Very Virulent IBD Virus (vvIBDv)

This study evaluated the efficacy of a one dose vaccine based on the combined administration of a live attenuated vaccine against an infectious bursal disease virus (IBDV) with a live attenuated Eimeria vaccine, when they are mixed prior to the administration by in ovo route to embryonated chicken eggs, against a challenge with a very virulent IBDV strain at day 24 of life.

A total of 28 embryonated chicken eggs from a commercial hatchery free of anticoccidials were chosen for this study. The eggs were randomly assigned into 2 different groups of 14 embryonated eggs each. At day 18 of incubation the embryonated eggs were injected by in ovo route with 0.05 ml/egg of the following vaccines according to the group assignment:
Group 1: Fourteen 18-day-old embryonated chicken eggs received a combined vaccine based on a combination of live attenuated Eimeria vaccine of the invention and a live attenuated Infectious Bursal Disease vaccine. The Eimeria vaccine comprised a mixture of attenuated Eimeria acervulina, E. maxima, E. praecox and E. tenella oocysts as follows: between 598-809 of sporulated oocysts from E. acervulina strain 120718P5, between 352-476 of sporulated oocysts from E. maxima strain 013, between 235-317 of sporulated oocysts from E. praecox strain 007 and between 221-299 of sporulated oocysts from E. tenella strain 004 per dose in phosphate-buffered saline solution (PBS) with 0.01% (v/v) of polysorbate 80. The live Infectious Bursal Disease (IBD) vaccine was GUMBOHATCH® (Laboratorios HIPRA S.A., Avda. La Selva 135, 17170 Amer, Girona, Spain).
   GUMBOHATCH® is a live immune complex vaccine comprising a minimum dose of 10^{1.48} PU (Potency Units) of live attenuated IBDV strain 1052 and 2.7×10⁶ VNU (Virus Neutralisation Units) of IBDV-specific antibody solution.
   GUMBOHATCH® is available on the European Union through the marketing authorisation issued by the EMA (EMEA/V/C/004967) on Dec. 11, 2019. It is also available in other non-European countries through the corresponding national licenses. Other IBD vaccines based on live immunocomplexes is CEVAC TRANSMUNE® (Ceva Animal Health, S.A., 08028 Barcelona, Spain) which is also available on the EU market among other markets.
   The combination vaccine of the present invention was prepared as follows: before the in ovo vaccine administration, one vial of lyophilized IBD-immunocomplex vaccine of 2000 doses (GUMBOHATCH®, Laboratorios HIPRA, S.A., Avda. La Selva 135, 17170 Amer, Girona, Spain) was reconstituted in 4 ml of sterile PBS, injecting the diluent into the lyophilized vaccine fraction. After 5 minutes, the composition was agitated to dissolve the product thoroughly. Once reconstituted, 0.103 ml of the IBD-immunocomplex vaccine was mixed with 8.877 ml of PBS and 1.020 ml of the attenuated Eimeria vaccine, previously mixed. The vial was shaken again before use.
   From this final volume of 10 ml a single dose of 0.05 ml of the mixed vaccine (Eimeria vaccine+IBD vaccine) was administered to each embryonated egg, before two hours after reconstitution.
Group 2: Fourteen 18-day-old embryonated chicken eggs served as control group. Animals in this group received a mock-vaccine comprising only PBS.

During the vaccination period, vaccinated and control chicken embryonated eggs and hatched chicks were handled identically, except for product exposure. In order to avoid contaminations between groups, the vaccinated and the control groups were clearly identified. All operations were performed first in the control group (Group 2) and thereafter in the vaccinated group (Group 1). Clinical signs were monitored at flock level. Litter and fresh faeces oocyst counts were also performed.

Before challenge, animals were sampled for blood in order to check antibody levels against IBDV, to confirm the correct vaccination with GUMBOHATCH® and to demonstrate that the control group remained seronegative.

For the evaluation of the efficacy against IBDV of the combined use of an IBDV vaccine and an attenuated Eimeria vaccine by in ovo administration, all hatched chicks of both Groups, 1 and 2, were experimentally infected at 24 days of age by the oculonasal route with 0.2 ml of a very virulent pathogenic strain of IBDV, strain VG-248 at a titre of 10^{6.4} EID₅₀/ml.

At day 6 after challenge (Day 30 of study), when the acute phase of the IBDv infection was expected to occur, animals were necropsied and the outcome of the infection was compared between groups. To assess the protection, bursas of Fabricius (BF) and spleens of the animals were examined macroscopically. A complete histopathological analysis of BF was performed based on Sharma et al., Comparative pathogenesis of serotype 1 and variant serotype 1 isolates of an infectious bursal disease virus and their effect on humoral and cellular immune competence of specific-pathogen-free chickens, Avian dis., 1989, 33:112-124.

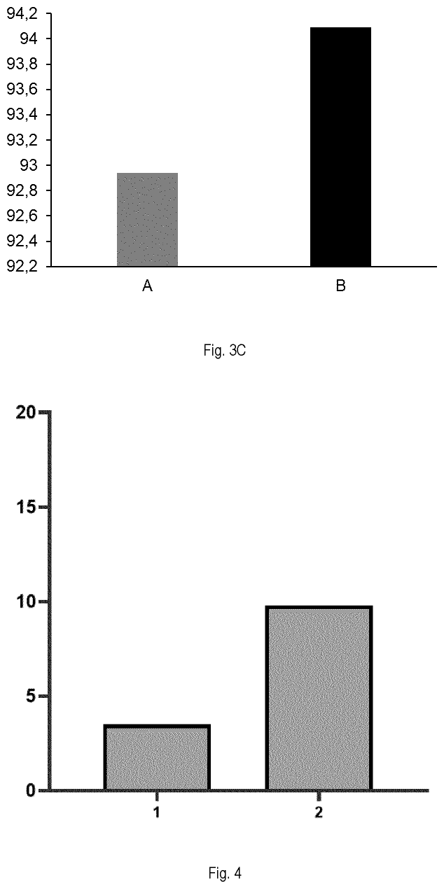

The acute histological functional lesions score was calculated per each animal and the mean per each group is represented in FIG. 4. The acute histological functional lesions score is the summary score obtained with the sum of individual scores on heterophil and mononuclear infiltration, haemorrhage, luminal exudate, plical oedema, oedema of the muscular wall, serosal oedema, necrotic cysts and lymphoid necrosis, which are the clinical parameters indicating acute lesions.

It is observed a significant reduction of the mean of acute histological functional score for vaccinated birds (Group 1) compared to the control group (Group 2). These results show that the combination vaccine is able to reduce acute functional lesions in the bursa in infected animals.

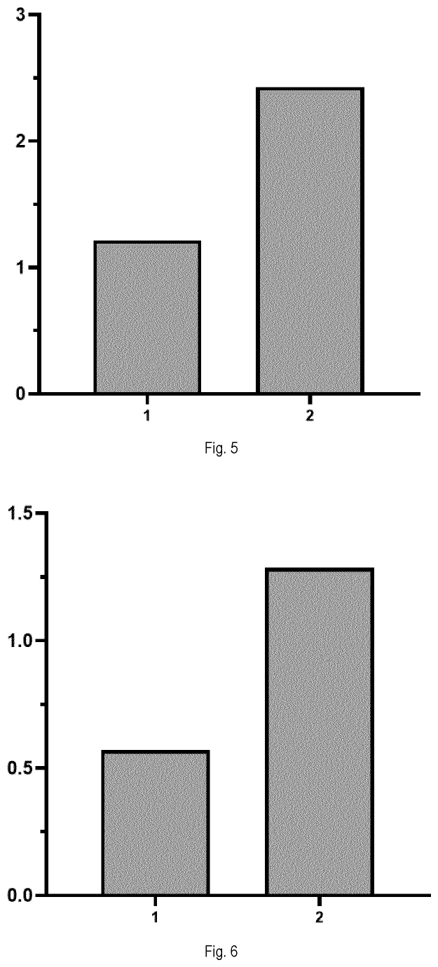

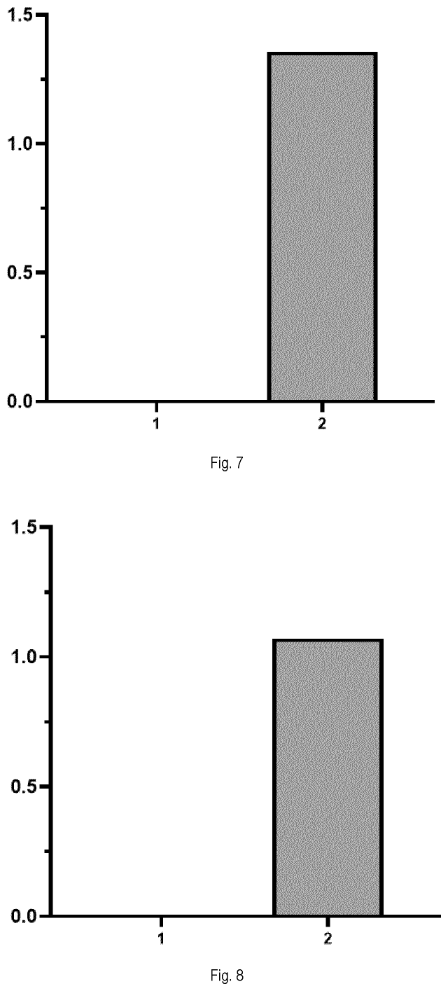

Furthermore, bursae from vaccinated group (Group 1) presented significantly lower inflammatory infiltrate and numerically lower plical oedema than animals in the control group (Group 2). Total absence of serosal oedema and oedema in the muscular wall was also observed in vaccinated group 1. This inflammatory response is indicative of the high virulent nature of the challenge strain, its absence in vaccinated animals means that the vaccine is able to not only induce an immune response but protect against very virulent IBDV strains such as the one used in the study (FIGS. 5-8).

Proportion of presence of external oedema in the bursae of Fabricius was also analysed for both groups. The presence of external oedema in the bursae of Fabricius is considered a clear sign of IBDv infection. External oedema was observed in 85.71% of the animals in the non-vaccinated control group (Group 2), whereas vaccinated animals in Group 1 did not show any clinical sign associated with external oedema at all, as shown in Table II:

TABLE II

| | Percentage of bird with external | |
|---|---|---|
| Group | oedema | |
| Group 1 (Combination vaccine) | 0/14 | (0.00%) |
| Group 2 (Control group) | 12/14 | (85.71%) |

All the differences observed between vaccinated group (Group 1) and non-vaccinated group (Group 2, control group) demonstrates that the combination vaccine clearly overcomes the effects of the vvIBDV, protecting the animals against vvIBDV infections and Gumboro disease.

Efficacy is demonstrated by the fact that appearance of serosal oedema, oedema in the muscular wall and external oedema in the bursae of Fabricius after challenge is fully prevented in vaccinated animals. Furthermore, vaccinated animals showed a reduction in the mean of acute histological functional score, inflammatory infiltrate and plical oedema compared to non-vaccinated animals.

Overall, the results obtained in this study surprisingly show that the combination of an attenuated Eimeria vaccine and an IBDV vaccine administered by in ovo route has no detrimental effects on the efficacy of the IBDV vaccine, while the combination vaccine is likewise efficacious against IBDV infection, thus protecting against Gumboro disease.

### Example 6: Efficacy of One Dose of the Combination of a Live Attenuated Eimeria Vaccine Against Avian Coccidiosis and a Live Attenuated Vaccine Against Infectious Bursal Disease (IBD) by in Ovo Administration to Embryonated Chicken Eggs Against a Challenge with Four Different Eimeria Spp

This study evaluated the efficacy of a one dose vaccine based on the combined administration of a live attenuated Eimeria vaccine when it is mixed with a live attenuated infectious bursal disease virus (IBDv) prior to the administration by in ovo route to embryonated chicken eggs, after a challenge with four different Eimeria species at day 21 of life.

A total of two-hundred and forty 18-day-old SPF embryonated chicken eggs were included in the study. The eggs were randomly assigned into 2 different groups of one-hundred twenty embryonated eggs each one. At day 18 of incubation the embryonated eggs were injected by in ovo route with 0.05 ml/egg of the following vaccines according to the group assignment:
Group 1: one-hundred and twenty 18-day-old SPF embryonated chicken eggs received a vaccine based on a combination of a live attenuated Eimeria vaccine of the invention and a live attenuated Infectious Bursal Disease vaccine. The Eimeria vaccine comprised a mixture of attenuated Eimeria acervulina, E. maxima, E. praecox and E. tenella oocysts as follows: between 598-809 of sporulated oocysts from E. acervulina strain 120718P5, between 352-476 of sporulated oocysts from E. maxima strain 013, between 235-317 of sporulated oocysts from E. praecox strain 007 and between 221-299 of sporulated oocysts from E. tenella strain 004 per dose in phosphate-buffered saline solution (PBS) with 0.01% (v/v) of polysorbate 80. The live Infectious Bursal Disease (IBD) vaccine was GUMBOHATCH® (Laboratorios HIPRA S.A., Avda. La Selva 135, 17170 Amer, Girona, Spain), already disclosed in Example 5. IBD disease is commonly known as Gumboro disease. The IBD virus gets permanently established in the poultry houses, becoming a resident pathogen infecting consecutive flocks and turning into a continuous threat.
   The combination vaccine used in this study was prepared as disclosed in Example 5. One vial of lyophilized IBD-immunocomplex vaccine of 2000 doses (GUMBOHATCH®, Laboratorios HIPRA, S.A., Avda. La Selva 135, 17170 Amer, Girona, Spain) was reconstituted in 4 ml of sterile PBS, injecting the diluent into the lyophilized vaccine fraction. After 5 minutes, the composition was agitated to dissolve the product thoroughly. Once reconstituted, 0.103 ml of the IBD-immunocomplex vaccine was mixed with 8.877 ml of PBS and 1.020 ml of the attenuated Eimeria vaccine, previously mixed. The vial was shaken again before use. From this final volume of 10 ml a single dose of 0.05 ml of the mixed vaccines (Eimeria vaccine+IBD vaccine) was administered to each embryonated egg, before two hours after reconstitution.
Group 2: One-Hundred and Twenty 18-Day-Old SPF Embryonated Chicken Eggs Served as Control Group. Animals in this Group Received a Mock-Vaccine Comprising Only PBS.

During the vaccination period, vaccinated and control chicken embryonated eggs and hatched chicks were handled identically, except for product exposure. In order to avoid contaminations between groups, the vaccinated and the control groups were clearly identified. All operations were performed first in the control group (Group 2) and thereafter in the vaccinated group (Group 1). Clinical signs were monitored at flock level. Litter and fresh faeces oocyst counts were performed.

Before challenge, animals were sampled for blood in order to check antibody levels against IBDV, to confirm the correct vaccination with GUMBOHATCH® and to demonstrate that the control group remained seronegative.

Then, the efficacy of the combined use of both vaccines was determined by means of an avian coccidiosis challenge model against each Eimeria species. included in the Eimeria vaccine of the invention. In this experimental model, each Eimeria infection was performed in a separate sub-study with an appropriate heterologous challenge Eimeria strain. Vaccinated and control birds were experimentally challenged by oral route at 21 days after hatching, according to Ph. Eur. requirements.

After challenge, for each Eimeria species, chickens were observed at least daily for 14 days. The following parameters were monitored: clinical signs (including bird's aspect and faeces appearance), mortality, intestinal lesions, oocysts count, weights and feed consumption. During this period, chickens were allocated in the same room for each Eimeria species and were raised in cages. All operations during the challenge period were performed blindly to the treatment.

The lesion scoring was done in a sample of animals of each group at day 4.5, day 6-, and day 7 post-challenge, which were the optimal times to perform macroscopical intestinal lesion analysis for E. praecox (4.5), E. acervulina and E. maxima (6) and E. tenella (7), respectively. Lesion scoring was done in accordance with the standard method of Johnson et al., op. cit., which is in line with the description contained in the European Pharmacopoeia monograph. For E. praecox, macroscopical lesions were evaluated according to the bibliographical reference of Williams et al. op. cit. Additionally, microscopical lesions were evaluated performing a histological study for E. praecox challenge test.

All procedures were approved by the Ethical Committee and fulfilled European regulations on animal welfare (2010/63/EU).

21 days after hatching, 30 birds per group were randomly assigned into 4 different challenge groups. Each group was experimentally infected (challenged) with different Eimeria species to assess the efficacy of the combination vaccine composition administered by in ovo route against avian coccidiosis as explained supra. Birds were experimentally infected by oral gavage administering 1 ml/bird of a preparation comprising oocysts from a virulent Eimeria strain, using an insulin syringe without needle. A first group was challenged with a preparation comprising 250,000 oocysts/ml from the virulent E. acervulina Houghton strain. A second group was challenged with a preparation comprising 100,000 oocysts/ml from the virulent E. maxima Weybridge strain. A third group was challenged with a preparation comprising 1,000,000 oocysts/ml from the virulent E. praecox Houghton strain. The fourth group was challenged with a preparation comprising 30,000 oocysts/ml from the virulent E. tenella Houghton strain. The distribution is described in Table III:

TABLE III

| | No. embryonated | No. hatched | |
|---|---|---|---|
| Treatment group | eggs | birds | Challenge species |
| 1 | 120 | 30 | E. acervulina |
| | | 30 | E. maxima |
| | | 30 | E. praecox |
| | | 30 | E. tenella |
| 2 | 120 | 30 | E. acervulina |
| | | 30 | E. maxima |
| | | 30 | E. praecox |
| | | 30 | E. tenella |

To assess the efficacy of the E. praecox strain 007 present in the combination vaccine composition administered by in ovo route, 18 birds per group were ethically euthanized on day 4.5 after receiving the experimental infection with the E. praecox Houghton strain, and intestinal lesions post-challenge were determined.

To assess the efficacy of the E. acervulina strain 120718P5 and E. maxima strain 013 present in the combination vaccine composition administered by in ovo route, 18 birds per group were ethically euthanized on day 6 after the experimental infection with E. acervulina Houghton strain and E. maxima Weybridge strain, and intestinal lesions post-challenge were determined.

To assess the efficacy of the E. tenella strain 004 present in the combination vaccine composition administered by in ovo route, 18 birds per group were ethically euthanized on day 7 after the experimental infection with E. tenella Houghton strain, and intestinal lesions post-challenge were determined.

Intestinal lesions were evaluated following Johnson et al. op.cit., for E. acervulina, E. maxima and E. tenella. For E. praecox the evaluation of lesions was performed following Williams et al. op.cit.

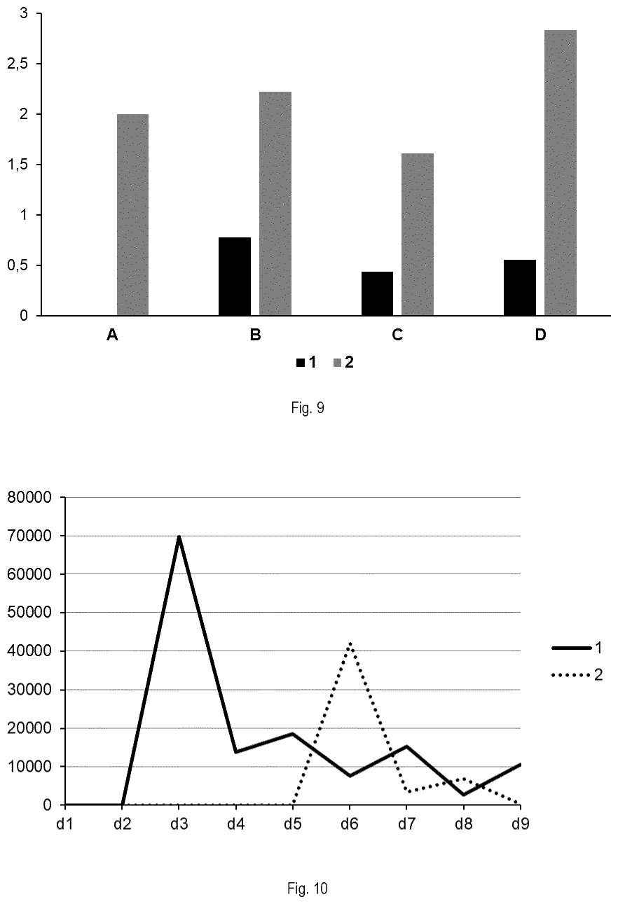

The mean of the intestinal lesion score per group and per experimental infection group assignment was then calculated. FIG. 9 shows the mean value of the intestinal lesion score per group and per experimental infection. Necropsies and lesion scoring were performed for each Eimeria species.

A significant reduction in the intestinal lesions in all groups that received the combination vaccine composition comprising sporulated oocysts derived from attenuated Eimeria strains and live attenuated IBDV (Group 1) was observed when compared to the non-vaccinated control group (Group 2). Furthermore, the combination vaccine resulted in the prevention of intestinal lesions caused by E. acervulina infection, as no intestinal lesions were observed at all.

The number of oocysts excreted in fresh faeces per bird in after experimental infection in each group were also assessed. Fresh faeces were recovered from trays from day 3 to 14 days post-challenge and the average number of oocysts excreted from fresh faeces per bird was determined. Results are shown in Table IV:

TABLE IV

| | Oocysts/bird | Oocysts/bird | |
|---|---|---|---|
| | faeces in | faeces in | |
| | Group 1 | Group 2 | % Reduction in |
| Experimental infection | (vaccinated) | (control) | vaccinated |
| E. acervulina | 4,365,694 | 223,128,519 | 98.0% |
| E. maxima | 2,842,700 | 22,332,741 | 87.3% |
| E. praecox | 607,215 | 15,840,741 | 96.2% |
| E. tenella | 5,487,852 | 71,899,190 | 92.4% |

Vaccinated group 1 which received the combination of the Eimeria and IBDv vaccine, shows that the administration of the combination vaccine in ovo to embryonated chicken eggs reduces significatively the excretion of oocysts after experimental infection for all different tested Eimeria species.

Oocysts excreted during the challenge period were reduced about 98.0% for E. acervulina in the vaccinated group compared to the control group, about 96.2% for E. praecox, about 92.4% for E. tenella and about 87.3% for E. maxima. Even, for E. maxima, the reduction in the percentage of oocyst count was significant.

Furthermore, 100% of vaccinated animals in group 1 seroconverted to IBDv at day 20, indicating that they were suitable vaccinated with the combination vaccine. Control animals remained negative to IBDV during all the vaccination period.

Therefore, a combination vaccine comprising oocysts derived from precocious attenuated strains selected from E. acervulina, E. maxima, E. praecox and E. tenella combined with live attenuated IBDV is able to generate an effective response against Eimeria infection and avian coccidiosis when administered by in ovo route to embryonated eggs. The effective response obtained with the combined vaccine of the invention reduces and/or prevents intestinal lesions and reduces the oocysts excretion caused by Eimeria infections.

Overall, the results of the study demonstrate the efficacy of the combination administration of one dose of a live vaccine against avian Infectious Bursal Disease and a live attenuated vaccine against avian coccidiosis, when mixed prior to be administered in ovo to 18-day-old embryonated chicken eggs, against avian coccidiosis caused by Eimeria infections.

Moreover, the parameters monitored during the vaccination period, prior to the experimental infection, were equivalent between the vaccinated group (Group 1) and the control group (Group 2). Thus, this further confirmed that the combination vaccine administered in ovo to embryonated chicken eggs has optimal safety.

### Example 7: Oocysts Elimination Profile

The main goal of this study was to assess the elimination profile of Eimeria spp. oocysts after the administration of live attenuated Eimeria spp. vaccine compositions administered by the in ovo route when compared to the administration of live attenuated Eimeria spp. vaccine compositions administered by coarse spray. The study was carried out in associated administration of additional antigens other than Eimeria spp. which were mixed prior to be administered in ovo to embryonated chicken eggs.

The Eimeria spp. vaccine composition used in this study is a live vaccine against avian coccidiosis comprising attenuated E. acervulina, E. maxima, E. praecox and E. tenella oocysts. Vaccines against Marek's disease (MD), Newcastle disease (ND) and infectious bursal disease (IBD) are routinely used in poultry farms. The concurrent use of Eimeria spp. vaccine compositions with other vaccines may result in interference since the in ovo vaccination is performed at a same time as are the other vaccines. Thus, it was also assessed whether these vaccines led to antigenic interference with Eimeria spp. vaccine compositions.

Vectormune® ND vaccine is a recombinant HVT vector vaccine, which uses the herpes virus of turkeys (HVT) as the vector and which genome the Fusion (F) gene of Newcastle disease virus (NDV) has been inserted. Vectormune® ND vaccine is indicated to be administered in ovo to 18-day-old chicken embryonated eggs or one-day-old chicks.

GUMBOHATCH® is a live immune complex vaccine indicated against Infectious Bursal Disease (IBD). The intended use of the vaccine is to protect chicks against very virulent IBD viruses (vvIBDV) once the maternal antibody levels start to drop. GUMBOHATCH® vaccine is also indicated to be administered in ovo to 18-day-old embryonated chicken eggs.

In this study, a total of two hundred and fourteen 18-day-old embryonated chicken eggs were included. The embryonated eggs were randomly assigned into 2 different groups of one-hundred seven eggs each one.

The first group of embryonated eggs (Group 1) received an in ovo injection of 0.05 mL/egg at 18 day of incubation of a vaccine based on a combination of a live attenuated Eimeria spp. vaccine composition, a live attenuated Infectious Bursal Disease vaccine and a recombinant Newcastle Disease Virus vaccine. The Eimeria spp. vaccine composition comprised a mixture of live attenuated Eimeria acervulina, E. maxima, E. praecox and E. tenella oocysts as follows: between 598-809 of sporulated oocysts from E. acervulina strain 120718P5, between 352-476 of sporulated oocysts from E. maxima strain 013, between 235-317 of sporulated oocysts from E. praecox strain 007 and between 221-299 of sporulated oocysts from E. tenella strain 004 per dose in phosphate-buffered saline solution (PBS) with 0.01% (v/v) of polysorbate 80.

The live Infectious Bursal Disease (IBD) vaccine was GUMBOHATCH® (Laboratorios HIPRA S.A., Avda. La Selva 135, 17170 Amer, Girona, Spain), already disclosed in Example 5 and 6. The recombinant Newcasle Disease Virus (NDV) vaccine was Vectormune® ND (CEVA-Phylaxia Co. Ltd., 1107 Budapest, Szállás u. 5, Hungary). Vectormune® ND is a live recombinant vaccine comprising a dose between 2500 and 8000 PFU (plaque forming units) of a recombinant turkey herpesvirus expressing the fusion protein of NDV. Vectormune® ND is available on the European Union through the marketing authorisation issued by the EMA (EMEAV/C/003829) on Aug. 9, 2015.

The combination vaccine was prepared as follows: 1 mL ampoule of Vectormune® ND was thawed following the vaccine's producer instructions of use and diluted in 42 mL of diluent. In this study the diluent was an aqueous solution comprising sucrose, casein hydrolysate, sorbitol, dipotassium hydrogen phosphate, potassium dihydrogen phosphate, phenol red and water for injection as described according to the EU marketing authorisation. Then, 6 mL of the live attenuated Eimeria spp. in ovo vaccine composition of the invention was slowly and gently added. After that, one vial of GUMBOHATCH® lyophilized vaccine was reconstituted in 4 mL of diluent. Finally, 1 mL of the reconstituted GUMBOHATCH® vaccine was added to the mix of 49 mL of Vectormune® ND and the live attenuated Eimeria spp. vaccine. A single dose of 0.05 mL of the mixed vaccine was administered in ovo per embryonated egg.

The second group of embryonated eggs (Group 2) received an in ovo injection of 0.05 mL/egg at 18 days of incubation of a vaccine based on a combination of a live attenuated IBD vaccine (GUMBOHATCH®) and a recombinant NDV vaccine (Vectormune® ND). The combination vaccine was prepared as follows: one ampoule of 2 mL Vectormune® ND was thawed following the vaccine's producer instructions of use and diluted in 96 mL of diluent. Then, 2 mL of the reconstituted GUMBOHATCH® vaccine was added. A single dose of 0.05 mL of the mixed vaccine was administered per egg.

Group 2 also received one standard dose of a live attenuated Eimeria spp. vaccine, EVANT® (Laboratorios HIPRA, S.A., Avda. La Selva, 135, 17170 Amer, Girona, Spain), at the day of hatch (1 day-old) by coarse-spray. EVANT® is a live attenuated Eimeria spp. vaccine comprising between 332-450 of sporulated oocysts from E. acervulina strain 003, between 196-265 of sporulated oocysts from E. maxima strain 013, between 293-397 of sporulated oocysts from E. mitis strain 006, between 293-397 of sporulated oocysts from E. praecox strain 007 and between 276-374 of sporulated oocysts from E. tenella strain 004 per dose. EVANT® is available on the European Union through the marketing authorisation issued by the EMA (EMEA/V/C/004902) on May 2, 2019.

Hatchability rates were recorded at hatch, and chickens were monitored for clinical signs and mortality at flock level. Moreover, litter and fresh faeces oocyst counts were performed to follow the vaccine elimination profile during the study. Blood samples were kept for serology studies regarding antibody response to IBD and ND as well as for qPCR test to HVT. Finally, weights of animals were also monitored during the trial. All operations during the duration of the study were performed blindly to the treatment.

Sequential necropsies were performed in order to study safety parameters. This evaluation included the study of the damage to the bursa of Fabricius and to evaluate intestinal lesions after the administration of the vaccines. Periodical necropsies were performed at days 21, 28 and 45 of the study. Twelve animals per group and timepoint were necropsied, so a total of 36 chickens per group were necropsied to study the safety. Blood samples were also collected at each time point to perform qPCR test to HVT. Body weight of all the animals was also recorded.

During the vaccination period, vaccinated and control chicken embryonated eggs and hatched chicks were handled identically, except for product exposure. In order to avoid contaminations between groups, the vaccinated and the control groups was clearly identified. All operations were performed first in the control group and thereafter in the vaccinated group.

To assess the Eimeria spp. oocysts elimination, samples of fresh faeces were recovered from each group on days 1 to 9 after hatch and the oocyst's output count was performed. The average Eimeria spp. oocysts counts in fresh faeces of each group at different days of study is shown in FIG. 10.

The results of this study demonstrate that the oocysts' elimination profile obtained after vaccinating the subjects by in ovo route with a live attenuated Eimeria spp. vaccine composition is different from the oocysts' elimination profile obtained after vaccinating the subjects with a live attenuated Eimeria spp. vaccine administered by coarse-spray.

Because the mechanism of live attenuated Eimeria spp. vaccines requires to complete Eimeria spp's live cycle within the host to properly immunize vaccinated subjects, the elimination of oocysts by vaccinated subjects is a key parameter to determine the protective performance of a live attenuated Eimeria spp. vaccine.

The study showed an unexpected earlier (on day 3) and higher (70000 oocysts' output/g fresh faeces) peak of oocysts' elimination profile for the in ovo live attenuated Eimeria spp. vaccine composition compared to the coarse spray administration (on day 6, 40000 oocysts' output/g fresh faeces). This indicated that the in ovo Eimeria spp. vaccine of the invention was able to initiate its life cycle earlier than the live attenuated Eimeria spp. vaccine administered by coarse spray.

Thus, the sooner the Eimeria spp. oocysts from live attenuated vaccines are eliminated, the sooner the immune response are triggered in the vaccinated subjects.

Therefore, the in ovo live attenuated Eimeria spp. vaccine composition of the invention is able to elicit an earlier immune response which in addition it was confirmed to be protective against Eimeria spp. infections in vaccinated subjects.

Moreover, the in ovo vaccination of Eimeria spp. vaccine compositions associated with currently commercial Marek, Newcastle and Gumboro vaccines did not affect neither the safety nor immunogenicity of the different antigens comprised in the vaccine compositions, so no antigenic interference was observed when combining such vaccines.

Similar results regarding the oocysts' elimination profile were also observed when the in ovo live attenuated Eimeria spp. vaccine composition was administered alone without combining it with further avian antigens such as Marek, Newcastle or Gumboro, confirming an earlier oocysts' elimination profile of the in ovo live attenuated Eimeria spp. vaccine composition over the Eimeria spp. vaccine administered by the coarse spray route.

What is claimed is:

1. A method for the prevention and/or treatment of an Eimeria infection, comprising administering in ovo to embryonated eggs of a domesticated bird a composition comprising Eimeria oocysts from a precocious attenuated strain of E. acervulina, and from at least one precocious attenuated strain selected from E. maxima, E. praecox and E. tenella.

2. The method according to claim 1, wherein the composition reduces or prevents hatched birds from one or more clinical signs associated with avian coccidiosis, wherein the clinical signs are selected from the group consisting of intestinal lesions, decreased body weight, oocysts output, oocysts load, oocysts shedding, diarrhoea, dehydration, presence of blood in faeces, presence of mucous in faeces, Eimeria transmission, depression, ruffled feathers, arched body position, morbidity, and mortality.

3. The method according to claim 1, wherein the method is for the prevention of avian coccidiosis.

4. The method according to claim 1, wherein the composition comprises a mixture of sporulated oocysts from precocious attenuated strains of E. acervulina, E. maxima, E. praecox, and E. tenella.

5. The method according to claim 4, wherein the E. acervulina precocious attenuated strain used therein, when administered to the host, the host excretes from about 30% to about 75% less of oocysts during a period of 14 days compared to the oocysts excreted by a host that received the parent E. acervulina strain.

6. The method according to claim 1, wherein the E. acervulina precocious attenuated strain used therein is E. acervulina strain 120718P5, deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021.

7. The method according to claim 1, wherein the composition comprises about between 100 and 6000 sporulated oocysts of E. acervulina per dose.

8. The method according to claim 1, wherein the composition is administered in a dose comprising between 0.050 ml and 0.20 ml.

9. The method according to claim 1, wherein the composition further comprises an additional avian vaccine, which comprises at least one immunogen, antigen or epitope of another avian pathogen.

10. The method according to claim 9, wherein the method is for the prevention and/or treatment of an Eimeria infection and Infectious Bursal Disease virus (IBDV, Gumboro disease) infection.

11. The method according to claim 9, wherein said composition is mixed with the additional avian vaccine prior to use and administered simultaneously in ovo to embryonated eggs of a domesticated bird.

12. The method according to claim 9, wherein the additional avian vaccine comprises at least one immunogen, antigen or epitope of avian pathogen of an infectious bursal disease virus (IBDV, Gumboro disease).

13. The method according to claim 12, wherein the method prevents and/or reduces at least one of clinical signs associated with Gumboro disease or IBDV infection selected from the group consisting of mean acute histological functional score, macroscopic lesions, mononuclear infiltration oedema, inflammatory infiltrate, plical oedema, absence of serosal oedema, and oedema in the muscular wall.

14. The method according to claim 1, wherein the composition is comprised in a kit.

15. An in ovo vaccine comprising Eimeria sporulated oocysts from a precocious attenuated strain of E. acervulina, and from at least one precocious attenuated strain selected from E. maxima, E. praecox and E. tenella, wherein the amount of sporulated oocysts from E. acervulina is about between 100 and 6000 per dose, with the proviso that the in ovo vaccine does not comprise E. mitis sporulated oocysts.

16. The method according to claim 1, wherein the method is for the prevention of avian coccidiosis and wherein the composition comprises a mixture of sporulated oocysts from precocious attenuated strains of E. acervulina, E. maxima, E. praecox, and E. tenella.

17. The method according to claim 1, wherein the composition comprises a mixture of sporulated oocysts from precocious attenuated strains of E. acervulina, E. maxima, E. praecox, and E. tenella, and the E. acervulina precocious attenuated strain is E. acervulina strain 120718P5, deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021.

18. The method according to claim 1, wherein the composition comprises a mixture of sporulated oocysts from precocious attenuated strains of E. acervulina, E. maxima, E. praecox, and E. tenella, and the E. acervulina precocious attenuated strain is E. acervulina strain 120718P5, deposited under the Budapest Treaty by HIPRA SCIENTIFIC, S.L.U. (Avda La Selva 135, 17170 Amer, Girona, Spain) in the Culture Collection of Algae & Protozoa (Scottish Marine Institute, OBAN, Argyl, PA37 1QA, Scotland, UK) under the accession number CCAP 2016/1 on Jun. 10, 2021, and wherein the composition comprises about between 100 and 6000 sporulated oocysts of E. acervulina per dose.

19. The method according to claim 1, wherein the method is for the prevention and/or treatment of an Eimeria infection and Infectious Bursal Disease virus (IBDV, Gumboro disease) infection, wherein the composition further comprises an additional avian vaccine comprising at least one immunogen, antigen or epitope of avian pathogen of an infectious bursal disease virus (IBDV, Gumboro disease).

20. The in ovo vaccine according to claim 15 further comprising an additional avian vaccine, wherein the additional avian vaccine comprises at least one immunogen, antigen or epitope of another avian pathogen.
